(19) United States
(12) Patent Application Publication
(10) Pub. No.: US 2013/0157285 A1
(43) Pub. Date: Jun. 20, 2013

(54) IDENTIFICATION OF INVASIVE AND SLOW-GROWING TUMORIGENIC CELL SUBSETS IN TUMORS

(71) Applicant: The Regents of the University of California, Oakland, CA (US)

(72) Inventors: Mandana Veiseh, Emeryville, CA (US); Eva A. Turley, London, CA (US); Mina J. Bissell, Berkeley, CA (US)

(73) Assignee: The Regents of the University of California, Oakland, CA (US)

(21) Appl. No.: 13/651,381

(22) Filed: Oct. 12, 2012

Related U.S. Application Data
(60) Provisional application No. 61/546,557, filed on Oct. 12, 2011.

(51) Int. Cl. C12Q 1/02

(52) U.S. Cl. CPC C12Q 1/02
USPC 435/7.1; 435/29; 536/55.1

(57) ABSTRACT

HA-based functional probes and a multiplexed targeting strategy for detection and isolation of invasive subpopulations in breast cancer cell lines. Methods for using HA metabolism for profiling and sorting breast cancer heterogeneity. As such, HA-based functional probes have appropriate targeting capacity and safety profiles for development as imaging and therapeutic agents for following repair and neoplastic disease processes such as breast cancer.

### CROSS-REFERENCE TO RELATED APPLICATIONS

This application is a non-provisional application and claims priority to U.S. Provisional Patent Application No. 61/546,557 filed on Oct. 12, 2011 and entitled “Identification of Invasive Tumorigenic Progenitor Cell Subsets in Tumors,” and is hereby incorporated by reference in its entirety. This application is also related to U.S. patent application Ser. No. 12/470,453 filed on May 21, 2009 and entitled “Rhamm, a Co-Receptor and Its Interactions with Other Receptors in Cancer Cell Motility and the Identification of Cancer Prognitor Cell Populations,” which is incorporated by reference in its entirety.

### STATEMENT OF GOVERNMENTAL SUPPORT

This invention was made with government support under Contract No. DE-AC02-05CH11231 awarded by the U.S. Department of Energy Office of Biological and Environmental Research and Low Dose Radiation Program; under Grant F32 CA132491 awarded by the National Cancer Institute of the NIH: under awards R37CA064786, U54CA126552, R01CA057621, U54CA112970, U01CA143233, and U54CA143836 awarded by the National Cancer Institute of the NIH; and the DOD-BCRP IDEA Grant W81XWH0810736 awarded by the U.S. Department of Defense. The government has certain rights in the invention.

### REFERENCE TO SEQUENCE LISTING, TABLE, OR COMPUTER PROGRAM APPENDIX

The present application refers to Tables 1-6 which are hereby incorporated by reference.

### BACKGROUND OF THE INVENTION

1. Field of the Invention

The present invention relates to tumor heterogeneity and metastasis, more specifically it relates to profiling cancer cell heterogeneity by glycosaminoglycans, identifying invasive subsets, and potentially progenitor cell subsets in tumors. It further relates to multiplexed detection of hyaluronan (HA) binding proteins and tumor progenitor markers at the surfaces of those tumor cell subsets. The present invention also relates more generally to: (1) diagnosis and prognosis of cancer by assessing tumor heterogeneity and identifying invasive subsets to select therapy regimens, and (2) therapeutics targeting those subsets.

2. Related Art

Breast tumors display substantial inter- and intra-tumoral heterogeneity driven by the interplay of genetic susceptibility, cellular lineage as well as hormonal and microenvironmental factors (Baumgarten and Frasor 2012; Boudreau, van't Veer et al. 2012; Marusyk, Almendro et al. 2012). These processes select for and support the appearance of tumor cell subpopulations with distinct phenotypes and functions such as proliferation rates, metastatic proclivity and treatment susceptibility. Regardless of breast tumor heterogeneity origin, identifying and targeting the functional compartment of malignant progression decision-makers impact the study and treatment of this disease. For instance identification of subpopulations that confer invasive behavior could improve therapy of advanced metastatic lesions and poorly-treated breast cancer subtypes such as triple-negative breast cancer, which still lack effective targeted therapy and have poor (˜20%) five-year survival rates (Metzger-Filho, O. et al. J Clin Oncol 30, 1879-1887, 2012)

Currently there is a paucity of biomarkers to identify BCa subpopulations (Liu, Li et al. 2012; Marusyk, Almendro et al. 2012; Metzger-Filho, Tutt et al. 2012). Those include isolation/surface characterization of normal stem cells for tracking tumor lineages (Petersen, Gudjonsson et al. 2003; Kim, Villadsen et al. 2012) and gene signature analyses (Dedeurwaerder, Fumagalli et al. 2011; Jeschke, Van Neste et al. 2012; Kabos, Finlay-Schultz et al. 2012; Pepin, Bertos et al. 2012; Prat, Ellis et al. 2012). Although detection of genetic heterogeneity may be a prognostic marker in BCa (Rakha and Reis-Filho 2009; Burrell, Juul et al. 2010; Irshad, Ellis et al. 2011; Marusyk, Almendro et al. 2012), the phenotypes manifested from this diversity are context dependent and therefore phenotypic rather than genotypic markers are more likely to provide the essential biological information required for design of therapies.

We set out to interrogate the heterogeneity of BCa cells based on measuring and selecting subpopulations that exhibit differential polysaccharide:cell interactions using the glycosaminoglycan, hyaluronan (HA) as a model. We reasoned this approach could reveal previously undetected heterogeneity linked to malignant progression because the diversity of tumor cells glycosylation patterns, which can occur as covalent and non-covalent modifications of proteins and lipids, is unrivaled (Potapenko, Haakensen et al. 2010; Rabinovich, van Kooyk et al. 2012) and more than other related polysaccharides, HA accumulation within BCa tumor cells and their microenvironment is associated with BCa progression and predicts poor outcome (ref).

HA accumulation in primary tumors (either stroma or tumor cells) is a predictor of poor outcome (Sironen, Tammi et al. 2011) and of conversion of DCIS to early invasive DCIS(Corte, Gonzalez et al. 2010). HA is produced by 3 distinct synthases and, unlike protein synthesis, is not strictly a template driven process. Rather it is subject to multiple synthetic steps, tight regulation and post-synthetic fragmentation by hyaluronidases and oxygen free radicals. As a result there are literally thousands of different HA sizes in remodeling microenvironments such as those of tumors (Potapenko, Haakensen et al. 2010; Veiseh and Turley 2011; Afratis, Gialeli et al. 2012; DeAngelis 2012; Erickson and Stern 2012), (Gao, Liu et al. 2010; Jiang, Liang et al. 2011; Tolg, Hamilton et al. 2012; Veiseh, Breadner et al. 2012) which bind differentially to HA receptors that are expressed on tumor cells (Jiang, Liang et al. 2011; Tolg, Hamilton et al. 2012; Turley and Naor 2012).

High accumulation of HA in the tumor and peri-tumor stroma is particularly associated with breast tumor progression as described by the inventors in Veiseh, M. and Turley, E., 2011. Integ. Biol. Advanced article. A method was developed for detecting cells that are actively metabolizing HA and using this method described herein that this property is shared by both normal but injured cells and aggressive BCA cell lines (FIG. 1, Table 1, and M. Veiseh, D. Breadner, N. Akentieva, R. C. Savani, R. E. Harrison, D. Mikilus, L. Collis, T. Y. Lee, J. Koropatnick, L. G. Luyt, M. J. Bissell, E. A. Turley. “Imaging of Homeostatic, Neoplastic and Injured Tissues by HA-based Probes.” Biomacromolecules, 13(1); 12-22, 2012, hereby incorporated by reference). Using a fluorescent HA probe, we showed that aggressive BCA lines such as MDA-MB-231 metabolized HA more actively than less aggressive BCA lines such as MCF-7 (FIG. 10). See also M. Veiseh, E. A. Turley. “Hyaluronan Metabolism in Remodeling Extracellular Matrix: Probes for Imaging and Therapy of Breast Cancer.” Integrative Biology, 3(4); 304-315, 2011, and M. Veiseh, D. Breadner, N. Akentieva, R. C. Savani, R. E. Harrison, D. Mikilus, L. Collis, T. Y. Lee, J. Koropatnick, L. G. Luyt, M. J. Bissell, E. A. Turley. “Imaging of Homeostatic, Neoplastic and Injured Tissues by HA-based Probes.” Biomacromolecules, 13(1); 12-22, 2012), and US PGPub US-2010-0062000-A1. This finding is consistent with previous evidence that MDA-MB-231 tumor cells produced larger amounts of HA, expressed higher levels of injury-induced HA receptors such as CD44 and RHAMM, (S. R. Hamilton, S. F. Fard, F. F. Paiwand, C. Tolg, M. Veiseh, C. Wang, J. B. McCarthy, M. J. Bissell, J. Koropatnick and E. A. Turley, The hyaluronan receptors CD44 and Rhamm (CD168) form complexes with ERK1,2 that sustain high basal motility in breast cancer cells, J. Biol. Chem., 282(22), 16667-80, 2007) and expressed higher levels of hyaluronidases.

Currently there are no effective technologies for the prevention and therapy of metastasis (N. Sethi, et al., Nature Reviews, 2011), partly due to late diagnosis and treatment of malignancy, as well as heterogeneity of metastatic colonies evolved from the same primary tumor. Tumor cells that are shed from the primary tumor disseminate throughout the body with characteristically different periods of latency and efficiency depending on tumor type or subtype (Fidler, I. J., Nature Rev. Cancer 3, 2003 and Nguyen, D. X., Nature Rev. Cancer 9, 2009).

### BRIEF SUMMARY OF THE INVENTION

The present invention provides for compositions, methods and assays using an HA probe to detect and accurately report sites of active HA metabolism which indicate the presence of invasive slow-growing cancer cells.

It was found that BCa cell lines differentially bound to a labeled HA probe (Tables 1 and 3). The highest HA probe binding was detected in highly invasive triple-negative “basal” subtypes such as MDA-MB-231, which currently do not have any positive marker for development of targeted therapies. BCa cell lines segregated to two distinct subpopulations based on low and high levels of binding (designated as HA^{-/low} and HA^{high}) which exhibited distinct invasive and growth properties. In a surrogate of metastatic and triple-negative breast cancer (ER−/PR−/HER2−) BCa subtype, high level of HA binding (HA^{high}) was associated with invasive phenotype of a minor subpopulation and unexpectedly, we found that HA^{high} subpopulations were proliferating slowly in culture and in vivo. Thus, these characteristics can be used for identification of aggressive cancers and isolation of the invasive cancer subsets within aggressive cancers. These invasive subsets may be identified and isolated from other types of cancers including, but not limited to, epithelial cancers, head and neck squamous cell carcinoma, prostate cancer, epithelial ovarian cancer, and endometriosis.

For physicians and healthcare providers, this technology presents a means to identify the highly metastatic triple-negative breast cancers. Ultimately, this technology could be used to sort patients needing invasive therapies from those who do not resulting in reduced patient morbidity and medical costs. In turn this sorting permits appropriate tailoring of therapeutic intervention for each patient based upon their risk for metastases. These results suggest that this method can be used to identify invasive tumor cell subsets that are likely resistant to current treatments. Current treatments target rapidly proliferating tumor cells, therefore the slow-growing subsets could escape treatment and increase the risk of peripheral disease since they are highly invasive.

The binding/uptake profile of the HA probe reveals a form of heterogeneity that is not detected by HA receptor displays. HA probe binding profiles reveal heterogeneous subsets spanning from 10¹ to 10⁵ binding intensities, and querying HA binding profiles in BCa lines shows highest heterogeneity in the most metastatic cell lines such as MDA-MB-231. Isolation of distinct subsets with at least 100 fold differences in HA binding (HA^{-/low} VS HA^{high}) identified invasive, yet slow-growing cell subsets that exhibited pathological signatures (i.e. lymphovascular invasion) of very advanced metastatic breast cancers. These results may aid diagnosis and therapy of invasive BCa subpopulations and early sorting of cancer patients susceptible to metastasis and needing intensive chemotherapy.

Current treatments target rapidly proliferating tumor cells, therefore the slow-growing subsets could escape treatment and increase the risk of peripheral disease due to metastasis. Moreover, slow-growing tumor cells (or dormant cancer cells) are nowadays accepted as major causes of cancer relapse and morbidity. The present methods could decrease patient morbidity and medical costs and increase effective clinical outcomes by early diagnosis of HA^{high} slow-growing subsets (i.e. the subset that binds to HA at least 100 times more than the rest of the cells). The methods presented in this invention is fast (HA metabolism is detected in less than 1 hr) and accurate.

The present probes and method may also be used to isolate CD44⁺/RHAMM⁺ BCA subpopulations and determined phenotypic and functional characteristics of isolated subpopulations. Results revealed that binding of the HA probe was heterogeneous in all molecular subtypes but was higher in basal than in luminal BCA lines, particularly in the highly-invasive subtypes exhibiting a CD44⁺/CD24^{-/low}/RHAMM⁺ surface phenotype. Isolation and growth of subpopulations that bound low levels of HA probe vs. high levels of HA probe showed stable differences in their 2-dimensional (2D)/3-dimensional (3D) culture morphology, surface markers, migration in culture, and tumorigenic properties in vivo. The CD44⁺/CD24^{-/low}/RHAMM⁺/HA^{high} subpopulation exhibited the most morphological heterogeneity in culture and in vivo, and contained a slow-growing subpopulation that aggressively invaded into the mammary fat pad and peritoneum. These results suggest that HA metabolism can be used to mark breast cancer heterogeneity, and that HA-based probes may ultimately be useful for imaging and for therapy of aggressive BCA subpopulations.

Thus, the present invention provides a HA-based functional probe and a multiplexed targeting strategy for detection and isolation of a slow-growing invasive HA-targeted subpopulation exhibiting a CD44⁺/CD24⁻/RHAMM⁺/HA^{high} phenotype. This CD44⁺/CD24⁻/RHAMM⁺/HA^{high} phenotype can be used as a marker of invasive subsets. The CD44⁺/CD24⁻/RHAMM⁺/HA^{high} subpopulation was initially poorly adherent and slow-growing and indicated the highest morphological heterogeneity and propensity for metastasis in 3D culture and in vivo.

The present invention further provides for detection, separation, therapy and imaging of highly-invasive but slow growing subsets of cells distinct from the rest of the dividing population of cells in a tumor. Thus, the potential of early sorting of patients needing chemotherapy or other aggressive cancer treatments from other types of cancers. Increased HA binding and uptake also likely provides a platform for additional and expanded therapeutic targets and therapies.

### BRIEF DESCRIPTION OF THE DRAWINGS

FIG. 1 (A) shows profiles of HA binding, which is heterogeneous (1 and 4), and both CD44 and RHAMM receptors (2 and 3) that are involved in HA binding. (B) shows fluorescent image of MDA-MB-231 after exposure to TR-HA in 2D, indicating that HA uptake is heterogeneous. (C) shows scanning electron micrographs (SEM) of MDA-MB-231 cells cultured in 3D indicating that HA-treated cells (left) form nano-aggregates on their surfaces.

Table 1 shows comparison between HA binding, HA receptor display, molecular subtype and phenotypes in human BCA lines.

FIG. 2 shows that HA binding reverts with reversion of MDA-MB-231 cells (A) to a reverted phenotype (B), and results in a significant reduction of the HA-binding positive population. Graphs indicate HA-binding FACS spectra of the cells after exposure to HA. Images indicate MDA-MB-231 cells (A) and reverted MDA-MB-231 cells (B) in 3D culture.

FIG. 3 shows that HA binding is heterogeneous in both cell types. HA-binding-based FACS method allows for the separation of viable cells into HA^{-/low} and HA^{high} subpopulations.

FIG. 4 shows HA^{-/low} subpopulations proliferate faster than the HA^{high} subpopulations and form larger and higher number of colonies (>5 fold difference).

FIG. 5 shows HA^{-/low} subpopulations are highly adherent, and form uniform spread and branched morphology in 2D and in 3D, respectively (left panel). HA^{high} subpopulations are poorly adherent (top right panel), form heterogeneous morphology, (i.e. round and spread morphology in 2D), and have compact and branched structures in 3D (right panel).

FIG. 6 shows that the HA^{-/low} subpopulation has lower motility than the HA^{high} subpopulation in a Boyden chamber assay.

FIG. 7 shows in vivo tumorigenic assessment of HA^{-/low} and HA^{high} subpopulations injected into intact mammary fat pads of female NOD-SCID IL2−/− mice. (A) tumor growth curve of the subpopulations. (B) ex vivo tumors before fixation (T:tumor).

FIG. 8 shows the histology of the tumors assessed in FIG. 7: (A) Discohesive epitheliod, (B) Spindle cell foci, (C) Peri-vascular invasion, (D) Necrotic foci, (E) Lymphovascular invasion, (F) Muscle invasion (Scale bars:100 μm,*:200 μm).

FIG. 9 (A) is a cartoon which shows how tumors may contain heterogeneous subpopulations with distinct surface phenotypes and functions. (B) is a cartoon which shows the pathway of how cell-surface RHAMM partners with CD44 in the presence of hyaluronan to activate ERK1/2, which results in the expression of genes required for motility and invasion (Maxwell, McCarthy et al. 2008).

FIG. 10 shows HA metabolism in scratch wounds (left images) and in breast cancer cell lines (right images). (A) Endogenous HA accumulates at the wound edge (arrows). (B) Texas Red-HA is also largely taken up at the wound edge, suggesting that sites of endogenous accumulation are also sites of active HA uptake/metabolism (arrow in right image of black/white panel). A similarly increased uptake of Texas Red-HA is observed in aggressive human BCA lines (e.g. MDA-MB-231 tumor cells), while much lower amounts of HA are taken up by less aggressive human BCA such as MCF-7 tumor cells. (Scale bar: 10 μm. Red color is Texas Red and blue is DAPI)

FIG. 11 shows (A) Identification and isolation of subpopulations based on HA binding level. (B) 2D growth and structure of HA^{-/low} vs. HA^{high} subpopulation in MDA-MB-231. (C) Colony forming evaluation of HA^{-/low} vs. HA^{high} subpopulation in MA-MB-231 cells (top) and SKBR-3 cells (bottom). (D) Growth and structure of HA^{-/low} vs. HA^{high} subpopulation in MA-MB-231 cells (top) and SKBR-3 cells (bottom) culture on Matrigel.

FIGS. 12 and 13 and Tables 2 and 3 show the quantification of histological observations in FIG. 8.

FIG. 14 is a schematic showing various aspects of using HA binding/uptake for functional and imaging assays, and diagnosis and prognosis.

FIG. 15 shows that CD44 mRNA expression is upregulated relative to normal breast tissue in triple negative (basal) but not luminal tumors, and that CD44/RHAMM co-expression in this subtype is linked to poor outcome in 80% of CD44+ve triple negative tumors.

FIG. 16 shows fluorescent HA (F-HA) binding to breast cancer cell lines is heterogeneous and is associated with malignant phenotype. (A) Schematic for fluorescent labeling of HA with hydrazide functionalized dyes. (B) Morphology of cells grown in 2D on glass culture substrata (scale bar xμm) prior to addition of F-HA. Graphs: HA binding profiles measured by FACS after addition of F-HA (+F-HA) to cells for 45 min. Dashed graph represents background fluorescent level of intact cells prior to addition of F-HA (−F-HA), which was similar for all cell types. (C) and (D) Comparison between HA binding levels of basal MDA-MB-231 and luminal SKBR-3 cells (measured by FACS geometric mean of positive binding) from 2 to 120 min after addition of F-HA, and after cell growth in laminin rich gels as 3D. Images: 3D morphology of MDA-MB-231 and SKBR-3 cells prior to F-HA addition. (E) HA binding profiles and morphology of MDA-MB-231 cells before and after reversion to non-malignant phenotype (by combined addition of AIIB2 and LY294002 within 48 hrs of culture).

FIG. 17 shows CD44 and RHAMM display profiles are non-Gaussian and uni-modal. Flow cytometry profiles of (A) CD44 and (B) RHAMM displays after incubating MDA-MB-231, T4-2, MCF-7, and SKBR-3 with anti-CD44 and anti-RHAMM antibodies, respectively.

FIG. 18 has real-time multiplexed analysis of HA/CD44/RHAMM interaction. (A) Background fluorescent level of control cells (i), RHAMM (ii) and CD44 (iii) expression levels. (B) Multiplexed analysis of the F-HA(i) after addition of RHAMM(ii) and CD44 (iii). (C) FACS analysis of F-HA binding profile at 4° C. and 37° C. (D) Fluorescent images of Texas red-HA uptake (top) and its cellular localization (bottom) (scale bar 50 μm). (E) TEM analysis of accumulated gold conjugated-HA in the nucleolus. (F) Fluorescence imaging of MDA-MB-231 treated with TR-HA(i), scrambled RHAMM (S-RHAMM) mimetic(ii), and RHAMM mimetic (iii).

FIG. 19 showed HA^{-/low} subpopulations grew more rapidly than HA^{high} subpopulations. (A) Schematic for identification and isolation of HA^{-/low} and HA^{high} subpopulations based on differential binding of cells to HA (markers indicate 8% selection) and their cell cycle modes immediately after isolation. (B) Lett. Morphology of HA^{-/low} subpopulations after adherence to 2D glass culture chambers within 2 days after sorting; Right. Morphology of HA^{high} subpopulations on 2D culture chambers within 2 days (round cells were non-adherent initially) and 7 days after sorting. Middle: growth status of subpopulations over 7 days.

FIG. 20 shows fluorescent HA signal disappears by 7 days.

FIG. 21 shows differential HA binding distinguishes functionally distinct cell subpopulations in MDA-MB-231. (A) Top. Top: A. colony forming ability HA^{-/low} (left) and HA^{high} (right) up to 13 days. (B) Colony forming ability of HA^{-/low} (left) and HA^{high} (right) from same number of cells on 3D soft agar by x days, (middle) quantification of (colony size or number have to check notes) by HA^{-/low} and HA^{high} subpopulations. (C) Morphology of HA^{-/low} (left) and HA^{high} (right) subpopulation on 3D culture (laminin-rich gels), (middle) quantification of occupied surface area by HA^{-/low} and HA^{high} subpopulations. (D) Quantification of HA^{-/low} and HA^{high} subpopulation invasion through matrigel-coated Boyden chambers as the average area occupied by cells that were passed through chamber.

FIG. 22 shows HA treated cells show increased migratory capacity in 3D culture and chorioallantoic membrane assays. (A) MDA-MB-231 cells expressing td Tomato red fluorescent protein were mixed with F-HA (blue) then immediately injected into veins in the chorioallantoic membrane (CAM) previously labeled with fluorescent Lens Culinaris Agglutinin to detect CAM vasculature (green). F binds to and is taken up by BCa and CAM endothelia. HA primarily accumulates the upper cell body over the nucleus and in cell processes that appear to be penetrating between endothelial cells. B(right). The side view is just the main cell body to show the HA within the cell more clearly. (B) Cell tracker tagged parental MDA-MB-231 cells extravasation from chick chorioallantoic membrane blood vessels (marked by red fluorescent lectin). (C) Intravital imaging of representative intravascular 231 tdT cells (left) and extravasated 231 tdT cells (right) 24 hr after injection of tumor cells (blood vessels marked by green fluorescent lectin). (D) Quantification of extravasatation efficiency following exposure to HA or control buffer at time 0 (T=0, top panels) and T=24 hrs. Image: number of untreated tumors cells and HA treated tumor cells present in the extracellular space (arrows mark examples of tumor cells).

FIG. 23 shows HA^{-/low} and HA^{high} subpopulations exhibit tumorigenic potential in NOD Scid IL2rgamma−/− mice, but the HA^{high} subpopulation gives rise to a slow-growing and invasive subset of tumors. Xenografts were formed in intact fat pads of 4^{th} mammary gland after injection of cells embedded in Matrigel. (A) Xenograft tumors derived from control, unsorted parental tumor cells (gray-dash), HA^{-/low} (black), and HA^{high} subpopulations (red); Inset: Gross morphology of excised tumors (T: tumor nodules). Wet weight measurements on the tumors derived from control, untreated parental tumor cells (gray), HA^{-/low} (black), and HA^{high} subpopulations (red). (B) Identification of 6 distinct pathological phenotypes by H&E staining of tumors and quantification of pathological observations (intensity bar indicates frequency of phenotype occurrence). (C) Staining patterns of CD44 in tumors derived from unsorted parental (control) and sorted tumor subpopulations (HA^{high} and HA^{-/low}).

Table 1 shows comparison between HA binding, HA receptor display, molecular subtype and phenotypes in human BCA lines.

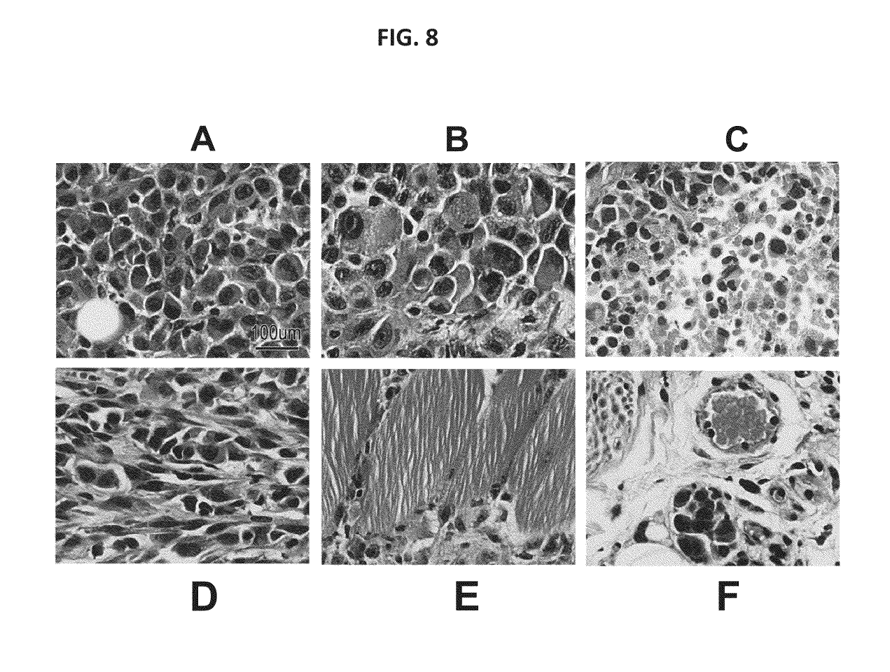

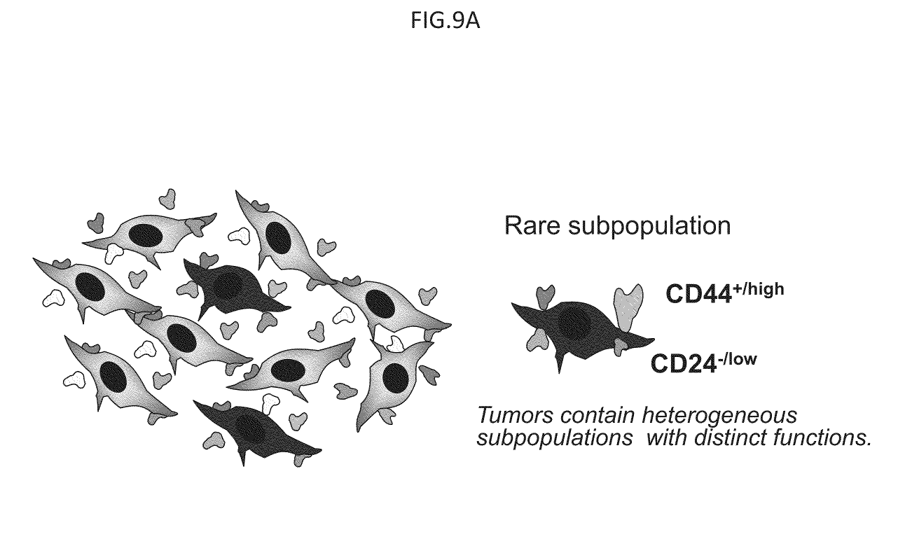

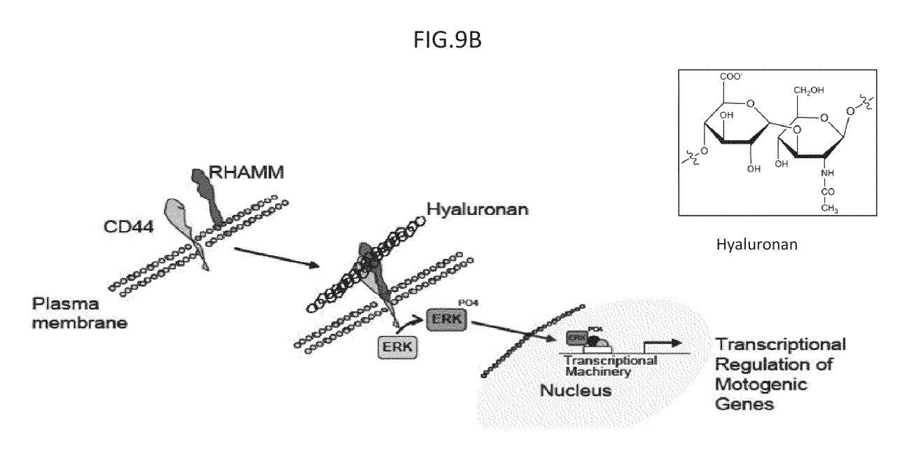

Table 2 and Table 3 show the quantification of histological observations in FIG. 8.

Table 4 lists characteristics of breast cancer cell lines

Table 5 shows confirmation that each cell line display CD44 and/or RHAMM

Table 6 shows a comparison between HA binding level and HA receptor display of human breast cancer cell lines.

### DETAILED DESCRIPTION OF THE PREFERRED EMBODIMENT

Herein are described a Hyaluronan (HA)-based functional probe and a multiplexed targeting strategy for detection and isolation of invasive subpopulations in breast cancer cell lines. It is shown herein that HA metabolism can be used for profiling and sorting breast cancer heterogeneity, based on a detectable marker of a five-fold increase in HA uptake by a cell. As such, HA-based functional probes have appropriate targeting capacity and safety profiles for development as imaging and therapeutic agents for following repair and neoplastic disease processes such as breast cancer. It is further contemplated that these invasive subpopulations of cells may also be identified and isolated from other types of cancers including but not limited to epithelial cancers, head and neck squamous cell carcinoma, prostate cancer, epithelial ovarian cancer, glioma, colon cancer, gastric cancer, leukemias, multiple myeloma, skin cancer, immune-cell related tumors, desmoids and other mesenchymal related tumors, endometriosis.

### DEFINITIONS

As used herein, the term, “aggressive” generally refers to a tumor or disease that forms, grows, or spreads quickly, and/or to cells that have tumorigenic potential which may exhibit tumorigenic characteristics including, but not limited to, increased cell division, proliferation, invasiveness, and/or motility, etc.

As used herein, the term “invasive” generally refers to cells or cancer that has spread beyond the layer of tissue in which it developed and is growing into surrounding, healthy tissues.

### DESCRIPTIONS OF THE EMBODIMENTS

In one embodiment, a method for detecting cells that are actively metabolizing HA and show high uptake of HA, which indicate a highly invasive and slow-growing population of tumorigenic cells. The high uptake of HA by these cells can be characterized by differential uptake of HA (e.g., about a five-fold increase in HA uptake. It was also observed that these cells exhibit a local invasion index of greater than 63%, where the local invasion index comprising the sum of perivascular invasion, lymphovascular invasion, and muscle invasion occurrence per cohort.

HA uptake and metabolism can be detected and quantified as the amount of labeled hyaluronan (HA), also referred to herein as HA probe, that can bind to and be taken up by cultured cells.

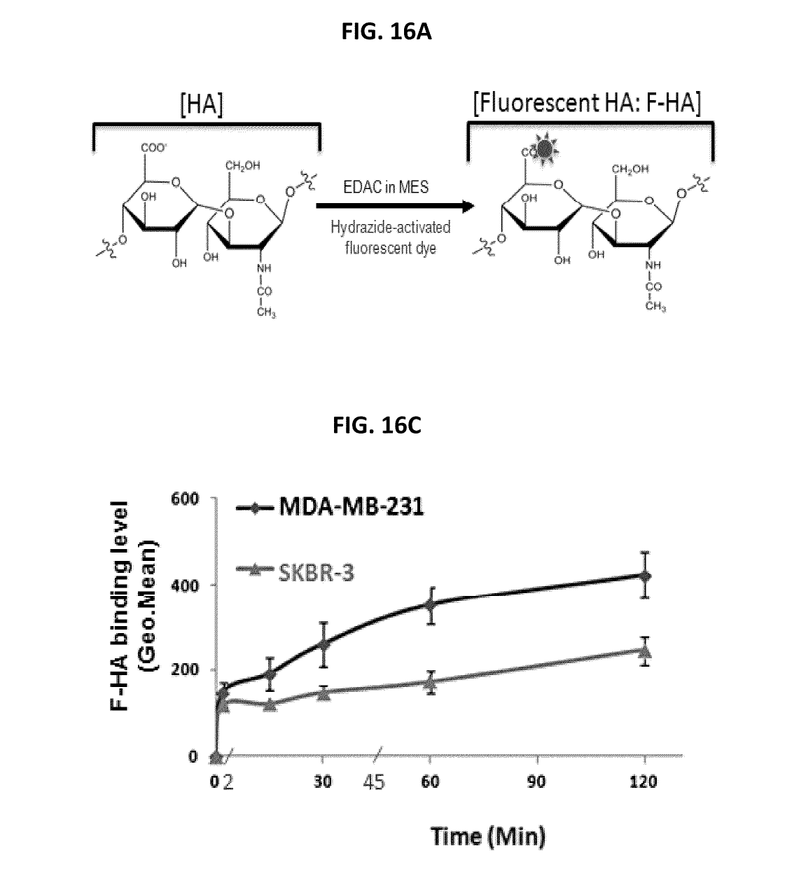

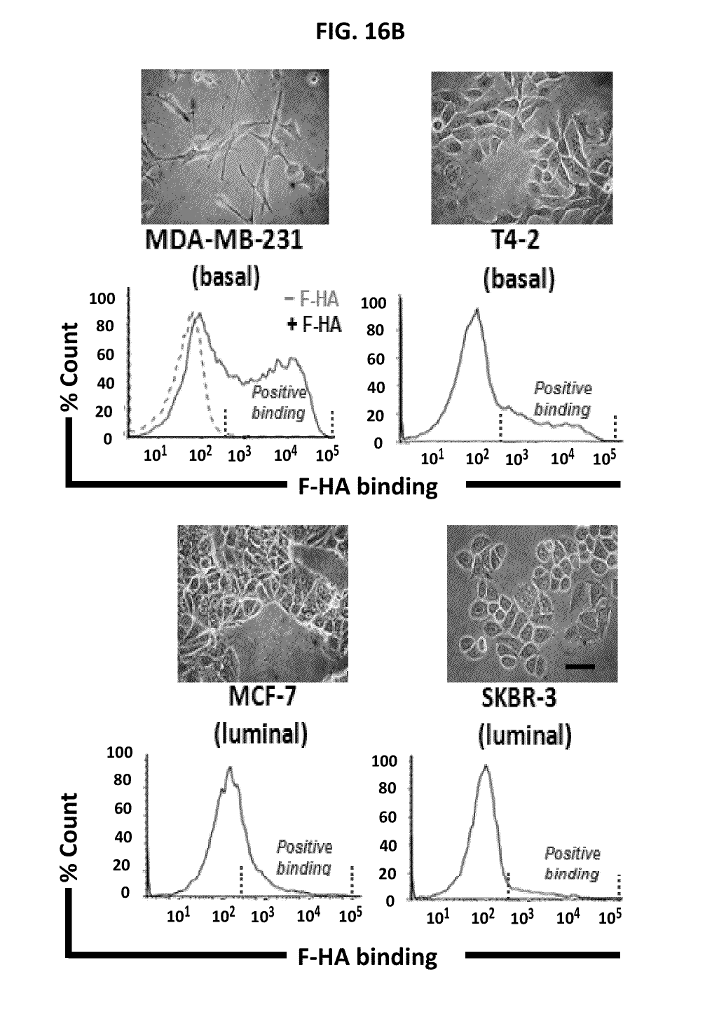

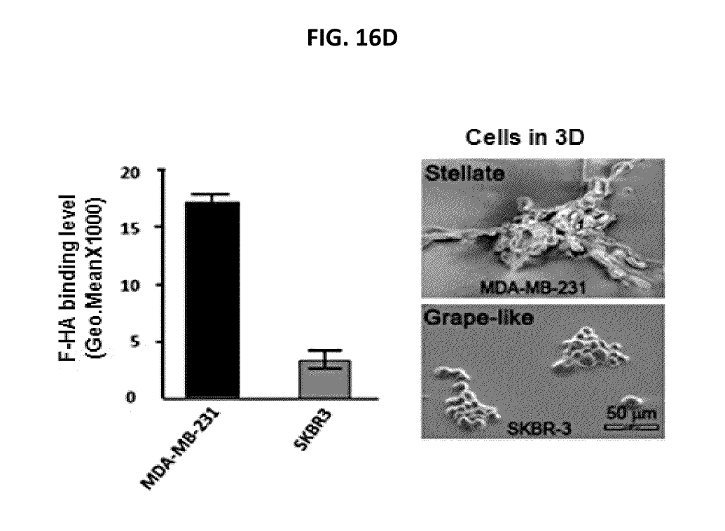

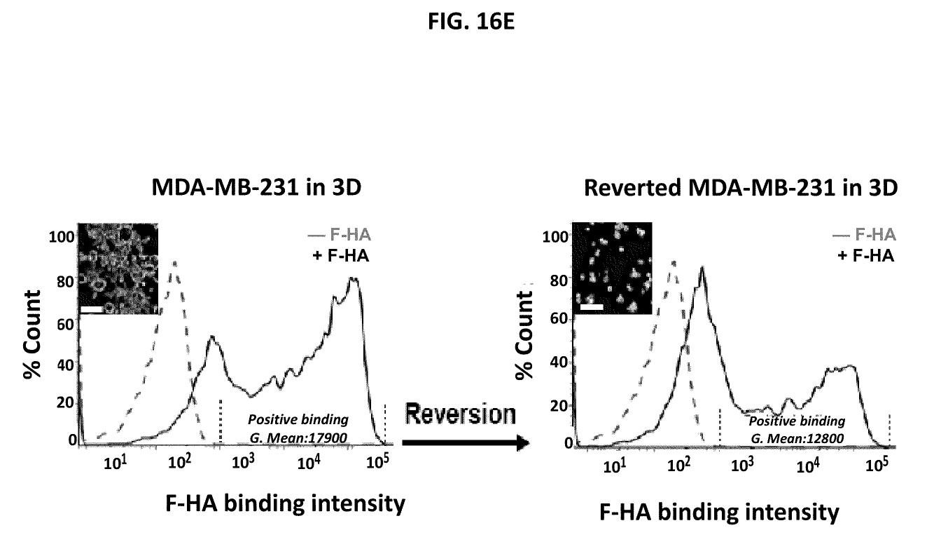

The HA probe can be made by direct attachment to a detectable label. In one embodiment, the conjugation can be as shown in the scheme in FIG. 16A. Thus no subsequent steps are required to associate the probe with the detectable label.

In some embodiments, the HA probe is a polydisperse (5-500 kDa Molecular weight range) labeled hyaluronan probe. In some embodiments, the HA probe is 280 kD or higher (anti-inflammatory) molecular weight. In other embodiments, the HA probe is lower than 280 kD in molecular weight. Further, in some embodiments, the HA probe is an HA fragment, polypeptides or a peptide mimetic. For example, H A peptides can include those described in Tolg C, Hamilton S R, Zalinska E, McCulloch L, Amin R, Akentieva N, Winnik F, Savani R, Bagli D J, Luyt L G, Cowman M K, McCarthy J B, Turley E A, A RHAMM Mimetic Peptide Blocks Hyaluronan Signaling and Reduces Inflammation and Fibrogenesis in Excisional Skin Wounds, Am J Pathol. 2012 October; 181(4):1250-70. doi: 10.1016/j.ajpath.2012.06.036. Epub 2012 Aug. 11; Turley E A, Naor D, RHAMM and CD44 peptides-analytic tools and potential drugs. Front Biosci. 2012 Jan. 1; 17:1775-94; Veiseh M, Breadner D, Ma J, Akentieva N, Savani R C, Harrison R, Mikilus D, Collis L, Gustafson S, Lee T Y, Koropatnick J, Luyt L G, Bissell M J, Turley E A. Veiseh M, Breadner D, Ma J, Akentieva N, Savani R C, Harrison R, Mikilus D, Collis L, Gustafson S, Lee T Y, Koropatnick J, Luyt L G, Bissell M J, Turley E A., Imaging of homeostatic, neoplastic, and injured tissues by HA-based probes, Biomacromolecules. 2012 Jan. 9; 13(1):12-22. Epub 2011 Dec. 12, all of which are hereby incorporated by reference.

The HA probe can be fluorescently labeled. In a preferred embodiment, multiplexed detection of CD44 and Rhamm is carried out by a fluorescent HA probe. Optionally, this probe may be labeled with a detectable marker to allow detection of the location of the invasive cancer subsets. Further, the detectable label allows tracing the movement of subsets.

Label materials include, but are not limited to organic and inorganic semiconductor materials, carbon, metals, and metal oxides. Suitable labels include but are not limited to fluorescent dyes (e.g., Texas Red, Alexa Fluor and Cy5.5 labels), radioactive labels (e.g., 125I, ³²P), metal (e.g., Au, Ag, Co, Ni, Pt), magnetic (e.g., GD, Fe2O3, TiO2 and the like), nanocrystals such as quantum dots of II-VI, III-V, and IV element groups (e.g., CdTe, GaP, SiO2,), and any mixtures of labels useful for multi-modal detection (e.g. magnetic-fluorescence). HA probe can be made in the form of nano- to micro-scale particles. In some embodiments the HA probe contains a biocompatible coating attached to it. In one embodiment, the HA probe is labeled with Texas Red or Alexa Fluor 647. In another embodiment, the HA probe can be made by direct attachment to a detectable label such that no subsequent steps are required to associate the probe with the detectable label.

In another embodiment, the HA probe is a mixture of HA of different molecular sizes.

The cells identified using the present methods can be obtained from a biopsy from a patient. The cells can be normal but injured cells, or suspected of being cancer cells.

In another embodiment, the cancer cell is in a mammal and further, that mammal is a human. In another embodiment, the cancer cell is biopsied from a subject. The cancer cell is contemplated to be any kind of cancer cell such as a breast cancer cell.

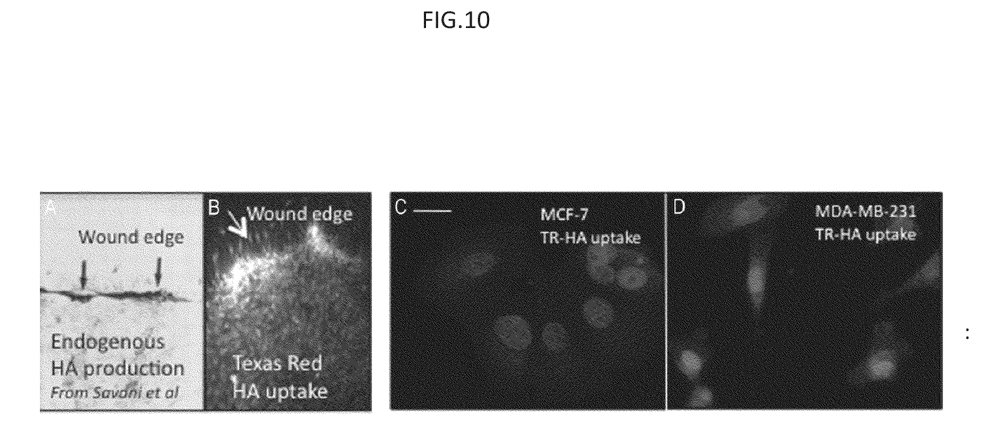

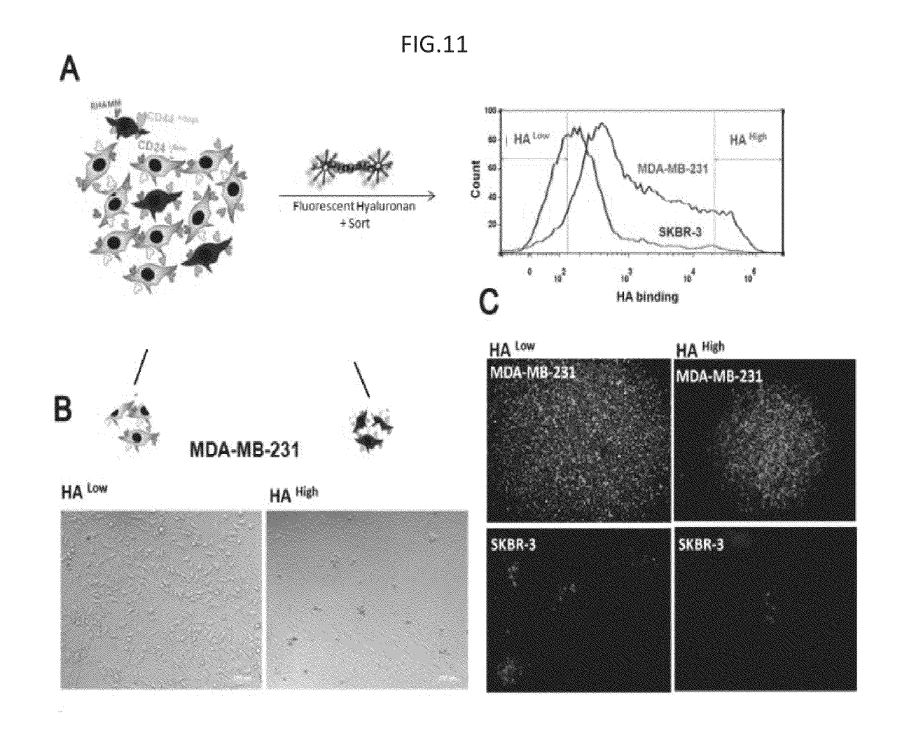

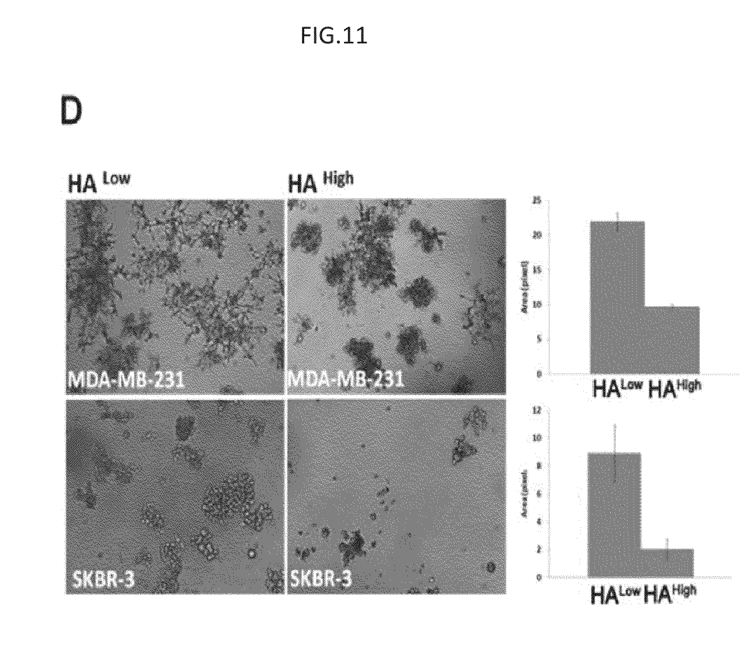

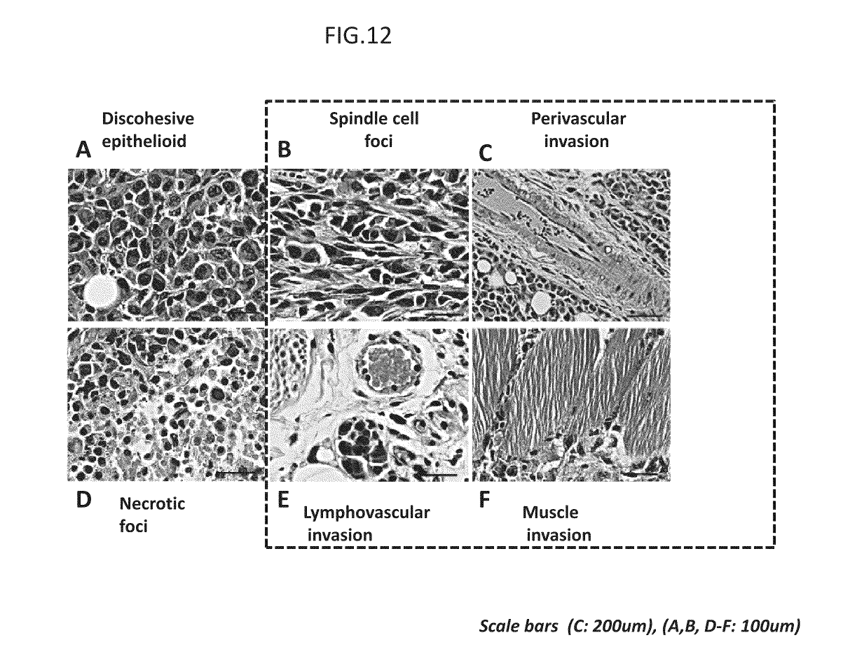

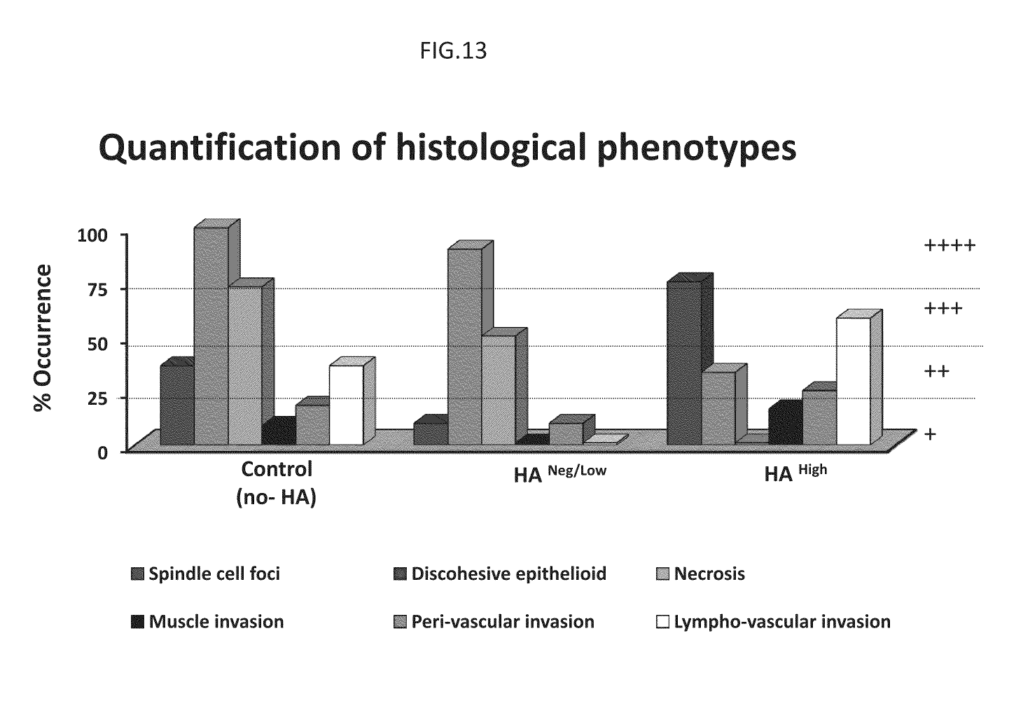

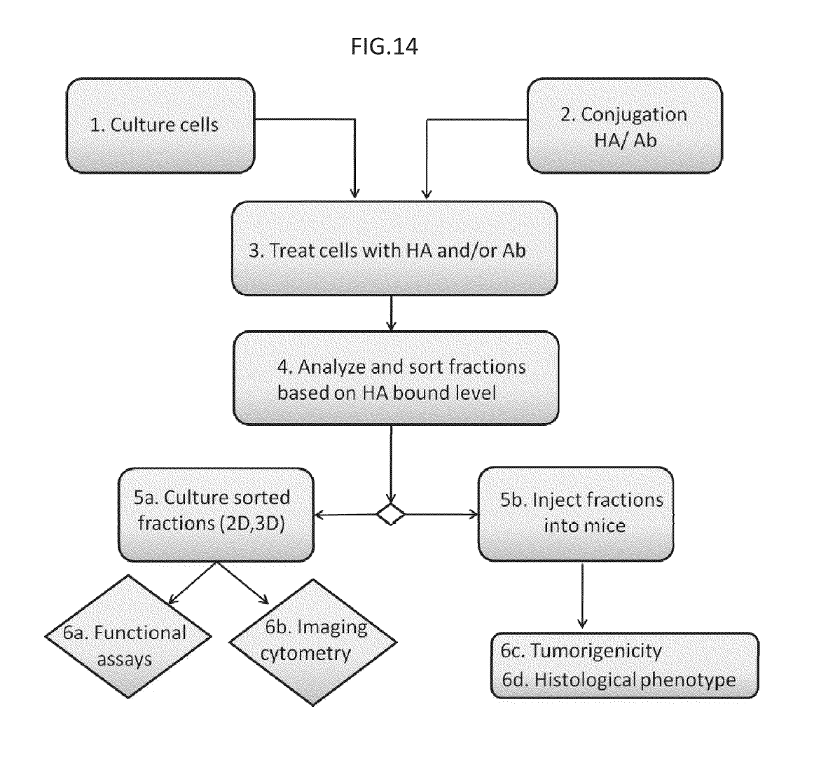

The biopsied cells can be normal but injured cells. For example, quiescent fibroblast monolayers showed little uptake, while scratch-wounding these monolayers resulted in high HA uptake at the wound edge (FIG. 10).

Using a fluorescently-labeled HA probe as a marker for high HA metabolism, we showed that aggressive BCA cell lines such as MDA-MB-231 metabolized HA more actively than less aggressive BCA lines such as MCF-7 (FIG. 10). This finding is consistent with previous evidence that MDA-MB-231 tumor cells produced larger amounts of HA, expressed higher levels of injury-induced HA receptors such as cluster designation 44 (CD44) and RHAMM (S. R. Hamilton, S. F. Fard, F. F. Paiwand, C. Tolg, M. Veiseh, C. Wang, J. B. McCarthy, M. J. Bissell, J. Koropatnick and E. A. Turley, The hyaluronan receptors CD44 and Rhamm (CD168) form complexes with ERK1,2 that sustain high basal motility in breast cancer cells, J. Biol. Chem., 2007, 282(22), 16667-80; (b) L. P. Wang, X. M. Xu, H. Y. Ning, S. M. Yang, J. G. Chen, J. Y. Yu, H. Y. Ding, C. B. Underhill and L. R. Zhang, Expression of PH20 in primary and metastatic breast cancer and its pathological significance, Zhonghua Bing Li Xue Za Zhi, 2004, 33(4), 320-3) and expressed higher levels of hyaluronidases. The results described in the Examples below show that an HA probe is able to functionally detect and accurately reports sites of active HA metabolism.

We next explored whether HA probes could selectively isolate CD44⁺/RHAMM⁺ breast cancer subpopulations and determined phenotypic and functional characteristics of isolated subpopulations. We quantified the HA probe binding profile and HA receptor display in a panel of human BCA lines that differed in molecular subtype and 3-dimensional (3D) morphological phenotype. Results revealed that binding of the HA probe was heterogeneous in all molecular subtypes, but was higher in basal than in luminal BCA lines, particularly in the highly-invasive subtypes exhibiting a CD44⁺/CD24^{-/low}/RHAMM surface phenotype. Isolation and growth of subpopulations that bound low levels of HA probe vs. high levels of HA probe showed stable differences in their 2D/3D culture morphology, surface markers, migration in culture, and tumorigenic properties in vivo.

The CD44⁺/CD24^{-/low}/RHAMM⁺/HA^{high} subpopulation exhibited the most morphological heterogeneity in culture and in vivo, and contained a slow-growing subpopulation that invaded into the mammary fat pad and peritoneum. These results show that HA metabolism can be used to mark breast cancer heterogeneity, and that HA-based probes may ultimately be useful for imaging and for therapy of invasive BCA subpopulations.

The HA^{Neg./low} population might also have important clinical impacts in the future as well because these cell populations have resisted HA probe binding/uptake. Thus detection of these cell subsets and profile are also contemplated.

The invasive but slow-growing subpopulation described herein is found in higher levels in basal than in luminal, particularly in highly invasive CD44+/CD24^{-/low}/RHAMM⁺ cell lines.

Thus, in one embodiment, a new marker combination of CD44⁺/CD24^{-/low}/RHAMM⁺/HA^{high} may be used as a marker for identification of tumorigenic progenitor cell subset. In another embodiment, a new marker combination of CD44⁺/CD24^{-/low}/RHAMM⁺/HA^{-/low} may be used for identification of fast-growing and HA^{low} binding subpopulations.

In another embodiment of the invention provides that the methods for prognosing cancer comprises providing a cancer cell and detecting the presence or absence of HA^{high}-binding subsets, whereby the presence of the HA^{high}-binding subsets indicates an aggressively metastatic cancer cell.

In yet another embodiment, the methods for prognosing cancer are performed in vivo by imaging the HA uptake in a subject.

In some embodiments, a method for measuring hyaluronan uptake and metabolism by a cell comprising the steps of: (a) providing a labeled hyaluronan probe (HA probe); (b) contacting a cell with the labeled HA probe; (c) detecting high HA uptake in said cell, wherein high HA uptake is at least 100 fold differences in HA binding and/or uptake and metabolism by said cell, which indicates said cell is highly invasive, slow-growing and able to invade the lymphovasculature, and (d) isolating said cell having high HA uptake.

In another embodiment, a method for the diagnosis and prognosis of a cancer patient comprising: (a) obtaining a tissue biopsy from a mammal, such as a human patient; (b) labeling cells from said biopsy with a labeled hyaluronan probe; (c) sorting said cells based on high or low uptake of the labeled hyaluronan probe; (d) determining if any cells highly uptake the labeled hyaluronan probe, wherein five-fold increased uptake of the labeled hyaluronan probe by cells indicates those cells are highly invasive.

In another embodiment, methods for the diagnosis and prognosis of a cancer patient were: (a) obtaining a tissue biopsy from a mammal, such as a human patient; (b) labeling cells from said biopsy with a labeled hyaluronan probe; (c) assessing said cells based on uptake profile of the labeled hyaluronan probe into cancer cells; (d) determining heterogeneity of the labeled hyaluronan probe uptake profile, wherein high heterogeneity index indicates those cells are highly invasive.

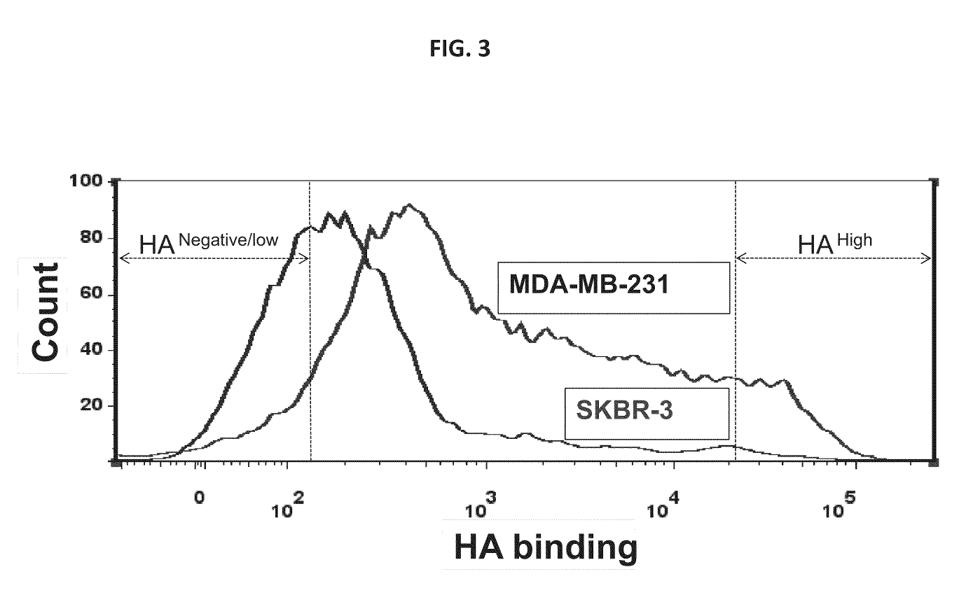

In one embodiment this invention provides a method for interrogating cancer cell heterogeneity by measuring and selecting subpopulations that exhibit differential polysaccharide: cell interactions using the glycosaminoglycan, hyaluronan (HA). In some embodiments, the HA uptake or binding profile is determined wherein a non-Gaussian HA binding profile, such as those shown in FIG. 3A, 11C, or 16B and 16E, indicates the detection of the highly-invasive slow-growing population of tumorigenic cells.

In one embodiment the profile of HA probe binding stratifies basal from luminal breast cancer subtypes.

In another embodiment the presence of these highly-invasive slow-growing subsets is detected by contacting sample with a probe (any formulations of HA or any chemical composition that behaves similar to HA) that specifically binds to HA, whereby the higher binding level and/or higher heterogeneity index indicates those cells are highly invasive. In one embodiment the invasive subsets can be targeted and eliminated by contacting sample with a probe (any formulations of HA or any chemical composition that behaves similar to HA) that carries drugs or inhibitors specifically to those subsets. Suitable molecules include, but not limited to, doxorubicin and cyclophosphamide, taxanes (paclitaxel, docetaxel), methotrexate, and 5-fluorouracil, epirubicin, inhibitor antibody, small molecule peptide, a siRNA, an antisense oligo, or aptamer.

In one embodiment, the present invention provides a method to identify and deliver therapy to triple negative breast cancer subtypes, which currently do not have any positive marker for development of targeted therapies.

In one embodiment, the present invention provides a method that permits identification of whether or not tumor (e.g., breast tumor) biopsies contain invasive subsets that can put the cancer patient at risk for developing metastases. The method comprises providing a sample and detecting the heterogeneity index and level of HA probe binding. Where the sample contains a high local invasion index and contains high amounts of HA-binding cell populations, this indicates that the sample contains invasive subsets and therapy specific for highly invasive albeit slow growing cells should be used to treat the patient. In another embodiment, a method for identifying invasive subsets in a patient is described herein. By identifying invasive subsets, the present method allows for the development of novel therapeutics to selectively kill these invasive cells. For example, this technology can be used for in vivo imaging of tumors that contain highly invasive subsets which allows for selective treatment of patients at risk for metastasis vs. those not at risk. Most importantly, this technology can be used to deliver anti-cancer drugs to the highly invasive subsets present in primary breast cancers. It is important to note also that although hyaluronan can be used as the delivery vehicle/ligand, so could mimetics of HA including peptide or small molecule mimetics as well as peptide or small molecule mimetics of HA binding partners including CD44 or Rhamm.

Another embodiment of the invention provides a method for prognosing cancer. The method contemplates a use for assessing invasive subsets correlated to patient outcome, and allows clinicians to sort patients with primary cancers and identify those patients at risk for metastases and those patients whose cancer is not or less likely to metastasize. In turn this sorting permits appropriate tailoring of therapeutic intervention for each patient based upon their risk for metastases. This is an important development in the art, as currently breast cancer patients that present with tumors are often channeled through the same therapeutic regime as currently the best indicator of poor outcome is the very crude measure of primary tumor size.

Thus, one embodiment of the invention provides that the methods for prognosing cancer comprises providing a cancer cell and detecting the differential uptake of HA by a cell, whereby the detection of high uptake, e.g., five-fold increase in HA uptake as compared to a control or normal cell, indicates the presence of a slow-growing yet highly invasive metastatic cancer cell.

Development of diagnostics that are based upon the molecular characteristics of invasive but slow growing subtypes would offer the additional advantage of sorting.

Moreover, since lymphovascular invasive phenotype is a feature of inflammatory breast cancer, which is the most metastatic of all breast cancers and has the lowest cure rate, the present method could improve breast cancer cure rate by earlier and more precise diagnosis of metastatic phenotype. This would reduce patient morbidity and medical costs and increase effective clinical outcomes. Phenotypic rather than genotypic markers are more likely to provide the essential biological information required for the design of effective therapies.

Through biochemical, functional, and imaging studies we established that HA^{high} subpopulation of malignant basal breast cancer xenografts gave rise to invasive yet slow-growing subset of tumors. This permits earlier prediction of patients' susceptibilities to metastasis before their tumors grow aggressively. This is an important development in the art, as currently the best indicator of poor outcome is the very crude measure of primary tumor size. Furthermore, since currently breast cancer patients that present with tumors are often channeled through the same therapeutic regiment.

Our phenotypic analyses showed a non-adherent (floating) phenotype for HA^{high} subset. Given that tumor stem-progenitor cells are floating around in the bloodstream, our HA metabolism assay could mark tumor stem/progenitor subsets in patients. In fact this method of sort would be extremely useful for identifying the very invasive population in leukemia. Clinicians use flow cytometry and FACS routinely on actual patient samples to phenotype cells. If a patient is selected to receive stem cell therapy for Hodgkins lymphoma, clinicians sort through existing stem cells in bone marrow as well as peripheral stem cells. The present method would identify the invasive subsets that might not be detected otherwise. Currently there is only a very crude way to decide whether patient should undergo stem cell transplantation without which a patient would only live about 5 years. If the tumor is not below the diaphragm, clinicians advise a stem cell transplant assuming that it has not spread into the bone marrow (only by correlation). Thus tumor size is used as a prediction of whether the tumor is aggressive or not. The present method could significantly improve outcome because the method permits screening the floating tumor cells in blood that bind to a large amount of HA and are possibly tumorigenic and very invasive without possibly excluding them.

In other embodiments, the disclosure provides for methods to inhibit cancer cell metastasis by targeting cells that have high HA uptake in vivo. In one embodiment, the method comprises attaching a drug, inhibitor, small molecule, antibody, siRNA, a mimetic, a peptide, an antisense oligo, or an aptamer or other therapeutic agent to HA, an HA probe, fragment or mimetic. These highly invasive but slow growing cell subsets should disproportionately uptake HA and the attached therapeutic, thus effectively delivering the therapeutic directly to the specific cell subset that most invasive and lymphovasculature positive.

A method of inhibiting cancer cell metastasis, the method comprising: (a) providing a polydisperse (5-500 kDa Molecular weight range) labeled hyaluronan probe (HA probe) attached to a therapeutic; (b) administering the labeled HA probe-therapeutic to a subject; (c) detecting high HA uptake in a cell, wherein high HA uptake is at least 100 fold differences in HA binding and/or uptake and metabolism by said cell, which indicates said cell is highly invasive, slow-growing and able to invade the lymphovasculature.

This method could be adapted for clinical management of breast or other tumor patients by providing further (e.g. additional to the phenotype of HA uptake, CD44+, Rhamm+ denoting aggressive tumor subsets) evidence of tumor aggression. This method could also be used to identify which treatments are most effective in shrinking the tumors before treating the patient. Imaging progenitor cell populations in vivo also allows a clinician to non-invasively monitor tumor shrinkage, cell progression, metastasis and efficacy of administered therapies.

In another embodiment, it is contemplated that a third therapeutic component is linked, attached or conjugated to the presently described probes. For example, a known cancer therapeutic (e.g., an anti-ErbB2 antibody or an anti-Her2 antibody), alone or coupled with a carrier compound, can be attached to the HA mimetic as a therapeutic component. Delivery to the site of tumorigenic cell populations can be confirmed by imaging in vivo and detecting the labeled HA. In one embodiment, the labeled probe having a therapeutic is labeled differently than another probe having only the targeting and imaging component for contrast.

Further methods for imaging and therapy that may be used in the present application are also described in copending application Ser. No. 12/470,453 filed on May 21, 2009 and entitled “Rhamm, a Co-Receptor and Its Interactions with Other Receptors in Cancer Cell Motility and the Identification of Cancer Prognitor Cell Populations,” previously incorporated by reference in its entirety.

### Example 1

### Detecting Cells Actively Metabolizing Hyaluronan

We have developed a method for detecting cells that are actively metabolizing HA and have shown that this property is shared by both normal but injured cells and aggressive BCA cell lines (FIG. 10, and M. Veiseh, J. Zhang, R. C. Savani, R. E. Harrison, D. Mikilus, L. Collis, J. Koropatnick, L. Luyt, M. J. Bissell and E. A. Turley, “Imaging of homeostatic, neoplastic, and injured tissues by HA-based probes,” Biomacromolecules. 2012 Jan. 9; 13(1):12-22. Epub 2011 Dec. 12). HA metabolism was detected and quantified as the amount of fluorescent, high molecular weight (HMW) HA—referred to as Texas Red-HA that can bind to and be taken up by these cultured cells. Quiescent fibroblast monolayers showed little uptake while scratch-wounding these monolayers resulted in high TR-HA uptake at the wound edge (FIG. 10). We verified that the TR-HA probe accurately detected areas of high HA metabolism by demonstrating its rapid and HA-receptor dependent uptake in both liver, which is perhaps the most active of all tissues in metabolizing HA, (P. H. Weigel and J. H. Yik, Glycans as endocytosis signals: the cases of the asialoglycoprotein and hyaluronan/chondroitin sulfate receptors, Biochim. Biophys. Acta, Gen. Subj., 2002, 1572(2-3), 341-63), and injured carotid arteries (vs uninjured contra-lateral carotid artery used as a control), which also exhibited increased HA metabolism. ((a) R. C. Savani and E. A. Turley, The role of hyaluronan and its receptors in restenosis after balloon angioplasty: development of a potential therapy, Int. J. Tissue React., 1995, 17(4), 141-51; (b) P. W. Noble and D. Jiang, Matrix regulation of lung injury, inflammation, and repair: the role of innate immunity, Proc. Am. Thorac. Soc., 2006, 3(5), 401-4).

Using a labeled HA probe as a marker for high HA metabolism, we showed that aggressive BCA lines such as MDA-MB-231 metabolized HA more actively than less aggressive BCA lines such as MCF-7 (FIG. 10). This finding is consistent with previous evidence that MDA-MB-231 tumor cells produced larger amounts of HA, expressed higher levels of injury-induced HA receptors such as cluster designation 44 (CD44) and RHAMM (S. R. Hamilton, S. F. Fard, F. F. Paiwand, C. Tolg, M. Veiseh, C. Wang, J. B. McCarthy, M. J. Bissell, J. Koropatnick and E. A. Turley, The hyaluronan receptors CD44 and Rhamm (CD168) form complexes with ERK1,2 that sustain high basal motility in breast cancer cells, J. Biol. Chem., 2007, 282(22), 16667-80; (b) L. P. Wang, X. M. Xu, H. Y. Ning, S. M. Yang, J. G. Chen, J. Y. Yu, H. Y. Ding, C. B. Underhill and L. R. Zhang, Expression of PH2O in primary and metastatic breast cancer and its pathological significance, Zhonghua Bing Li Xue Za Zhi, 2004, 33(4), 320-3) and expressed higher levels of hyaluronidases. These results imply that the HA probe is functional because it detects and accurately reports sites of active HA metabolism. A number of studies indicate that HA metabolism is a transient but essential process for wound resolution ((a) R. D. Price, M. G. Berry and H. A. Navsaria, Hyaluronic acid: the scientific and clinical evidence, J. Plast., Reconstr. Aesthetic Surg., 2007, 60(10), 1110-9; (b) W. Y. Chen and G. Abatangelo, Functions of hyaluronan in wound repair, Wound Repair Regener., 1999, 7(2), 79-89; (c) K. R. Taylor and R. L. Gallo, Glycosaminoglycans and their proteoglycans: host-associated molecular patterns for initiation and modulation of inflammation, FASEB J., 2006, 20(1), 9-22; (d) D. Jiang, J. Liang and P. W. Noble, Hyaluronan in tissue injury and repair, Annu. Rev. Cell Dev. Biol., 2007, 23, 435-61; (e) M. Slevin, J. Krupinski, J. Gaffney, S. Matou, D. West, H. Delisser, R. C. Savani and S. Kumar, Hyaluronan-mediated angiogenesis in vascular disease: uncovering RHAMM and CD44 receptor signaling pathways, Matrix Biol., 2007, 26(1), 58-68; (f) M. I. Tammi, A. J. Day and E. A. Turley, Hyaluronan and homeostasis: a balancing act, J. Biol. Chem., 2002, 277(7), 4581-4). However, our results and those of others indicate that it is aberrantly and constitutively active in aggressive breast cancer and likely contributes to progression of this disease rather than its resolution ((a) R. H. Tammi, A. Kultti, V. M. Kosma, R. Pirinen, P. Auvinen and M. I. Tammi, Hyaluronan in human tumors: pathobiological and prognostic messages from cell-associated and stromal hyaluronan, Semin. Cancer Biol., 2008, 18(4), 288-95; B. P. Toole and M. G. Slomiany, Hyaluronan: a constitutive regulator of chemoresistance and malignancy in cancer cells, Semin. Cancer Biol., 2008, 18(4), 244-50)

### Example 2

### Detecting and Imaging Tumor Heterogeneity

Tumor heterogeneity is a characteristic of breast cancer (BCA) at both the molecular and histological level. The presence of heterogeneous tumor cell subpopulations, which exhibit distinct cell surface markers, biological functions and tumorigenicity, has recently been implicated in both BCA progression and response to treatment. In particular, increasing evidence implicates stem/progenitor-like subpopulations in BCA initiation, metastasis, and resistance to therapy. These particular subpopulations are characterized by high surface display of CD44, what is now considered to be the minimal surface phenotype amongst additional surface markers. CD44 is an integral receptor for the tissue polysaccharide, hyaluronan (HA), which activates the signaling functions of CD44 in BCA. Another HA receptor, RHAMM/HMMR, is a co-receptor for CD44 and promotes its ability to activate MAPK pathways through HA and HA fragments (See Tolg C, Hamilton S, Zalinska E, McCulloch L, Amin, R, Akentieva N, Winnik F, Savani R, Bagli D J, Luyt L G, Cowman, M K, McCarthy, J B and Turley E A. 2012. A RHAMM mimetic peptide blocks hyaluronan fragment signaling and promotes skin excisional wound repair by reducing inflammation. Am J. Pathol. 181: 1250-70; Toelg C, Hamilton S R, Kooshesh P, McCarthy J B, Bissell M J, and Turley E A. 2006. Rhamm^{-/-} mice are defective in CD44-mediated ERK1,2 mitogenic signaling, leading to defective skin wound repair. J. Cell Biol. 175:1017-28, all of which are hereby incorporated by reference). We hypothesized that stem/progenitor-like BCA subpopulations also overexpressed RHAMM protein and this elevated expression would result in a high rate of HA-based probe uptake/internalization. This would permit isolation of aggressive, tumorigenic subpopulations based on differential HA receptor (RHAMM CD44) display/HA uptake. The long-term goal was rational design of HA-based targeted imaging and therapeutic strategies for invasive BCA subpopulations. Development of targeting strategies based on the characteristics of invasive subtypes would offer the additional advantage of sorting patients into different treatment streams to reduce patient morbidity and medical costs, and to increase effective clinical outcome.

In this context, we defined two avenues of investigation: (I) understanding the biology of high HA uptake that contributes to breast tumor progression and (II) evaluating the efficacy of HA-based probes for BCA imaging or therapy. We developed (1) a near infrared fluorescent-HA probe and a multiplexed approach for accurate profiling of HA binding to the proposed cell surface markers in a large panel of breast cell lines, (2) culture assays that could distinguish tumor cell subpopulations based on their phenotypic behavior, and (3) micro-electromechanical systems (MEMS) platforms with tunable physical/chemical properties for high-resolution biochemical and biophysical characterization of subpopulations. Using these tools we identified and characterized two tumor subpopulations based on differential HA receptor display and HA binding in BCA cell lines as discussed in the results section. These results are currently being written up as manuscripts. In translational studies, we assessed the efficacy of HA for imaging probe development and evaluated/quantified detection of HA-based nanoprobes in BCA and response-to-injury animal models.

Development of a Method for Real Time Multiplexed Profiling of Cell Surface-Markers and Bound Hyaluronan.

Simultaneous profiling of cell surface markers and HA binding/uptake required the establishment of a sensitive multiplexed detection method, which could detect both the cell surface receptors and the binding/uptake of ligand (HA). We utilized fluorescence-activated cell sorting (FACS) and established protocols for minimum decoration of cells by antibodies and HA, maximum detection sensitivity at the cell surface, and retained viability/functionality during and after sort. Fluorescent HA probes and antibodies were synthesized and characterized in a panel of human breast cancer cell lines (MDA-MB-231, SUM1315MO2, HS-578T, MDA-MB-468, HCC-1569, BT-20, MCF-10A, S1, T4-2, MCF-7, BT474, SK-BR3, AU565, MDA-MB-361, D920, SK3 [RHAMM−/− and CD44−/− were used as negative controls], and 10T½ [RHAMM+/+ were used as positive controls]). In brief, soluble HA (280,000 dalton) was linked to hydrazide-activated fluorochromes and monoclonal antibodies were directly conjugated to tetrafluorophenyl (TFP) ester activated fluorochromes (Alexa Fluor 647 and sometimes to Alexa Fluor 488) and reacted with the live cells under sterile dark conditions. Four fluorochromes were selected in a manner that provided minimal fluorescent spectral overlaps and maximal brightness for low-expressing markers, which allowed sensitive monitoring of subtle changes in marker expression and HA binding capacity in live cells. Near infrared fluorescence range was selected for HA to extend signal stability during multiple stages of characterizations (Alexa Fluor 647). We also prepared conjugated quantum dots, but their interaction dynamics and kinetics with cells did not match the fluorochrome conjugated materials. As well, these appeared to non-specifically interact with each other and with cells, which would require extensive characterization and optimization. Therefore, we continued our study using minimally-decorated HA and antibodies by fluorochromes, as described below, and successfully profiled multiple cell surface markers.

HA Binding Sensitivity and Specificity Assessment in Breast Cancer Cell Lines.

We quantified the HA probe binding/uptake profile, HA receptor display, cancer stem-like surface markers, and the percentage of cells which were CD44⁺/CD24^{-/low}/Rhamm⁺/HA^{high} or CD44^{-/low}/CD24⁺/Rhamm⁺⁺/HA^{high} in a panel of human BCA lines that differed in their molecular subtype, tumor phenotype and 3-dimensional morphological phenotype (Table 1). We selected the following lines from the larger panel as representative of clinically important molecular and functional signatures that are associated with differences in HA probe binding/uptake.

Our analyses confirmed the results obtained by Sheridan, C. et al. showing a surface phenotype of CD44⁺/CD24^{-/low} for MDA-MB-231, and CD44^{low}/CD24⁺ for MCF-7 and CD44⁻/CD24⁺ SKBR-3. HA binding profiling and cell-surface marker analysis additionally revealed that overall HA binding was heterogeneous and higher in basal than in luminal breast cancer cell lines, particularly in the most invasive basal subtypes that exhibited a CD44⁺/CD24^{-/low}/RHAMM⁺ phenotype. Subsets of luminal BCA also exhibited a CD44^{-/low}/CD24⁺/RHAMM⁺⁺ surface display, but bound HA to a lesser degree than basal lines. In these lines, higher probe uptake was most strongly related to surface RHAMM expression (Table 1).

These HA-binding profiles were intrinsic and stable to the BCA subtype and did not appear to be a function of culture conditions or culture temperature. Comparisons were made between fluorescent intensity (measured by FACS geometric mean) in HA-bound subpopulations of different cell lines that were either grown as 2D on plastic or 3D on matrigel. Regardless of culture condition, MDA-MB-231 showed higher HA binding/uptake than SKBR-3, and, interestingly, the binding differences were more pronounced in 3D than in 2D-grown cells. Kinetic analysis up to signal saturation indicated that HA binding differences remained robust over time (data not shown).

HA Binding is Receptor Mediated.

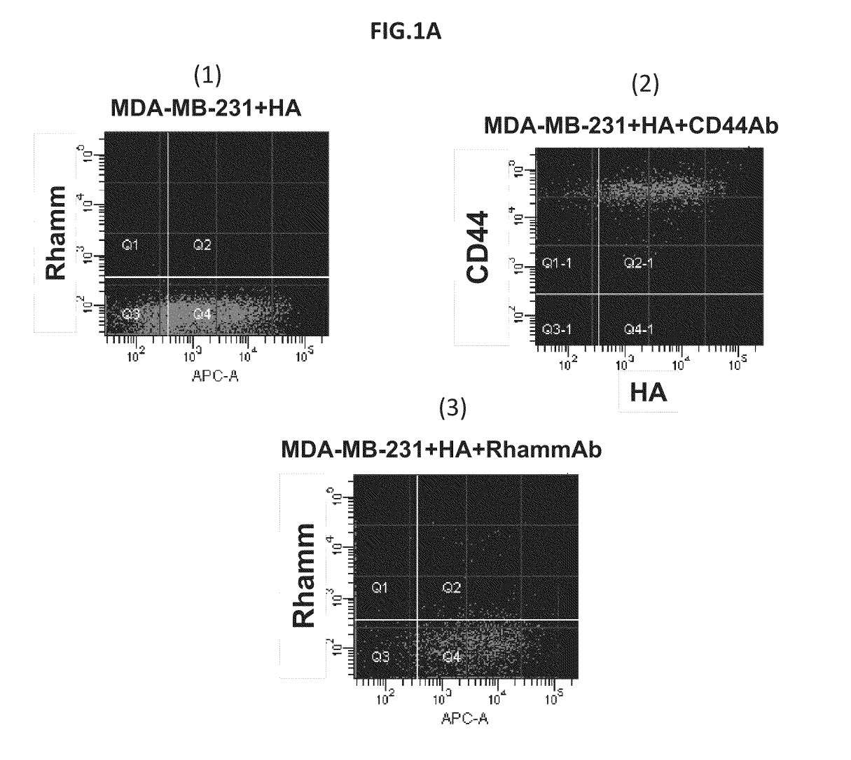

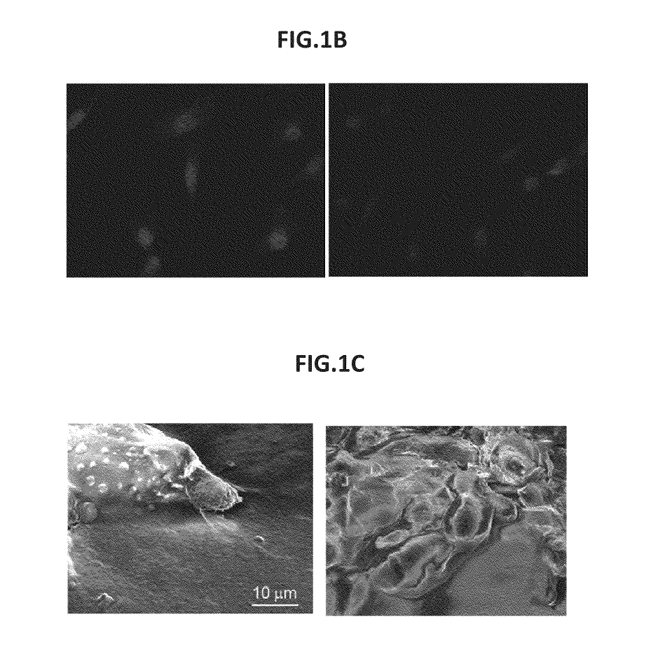

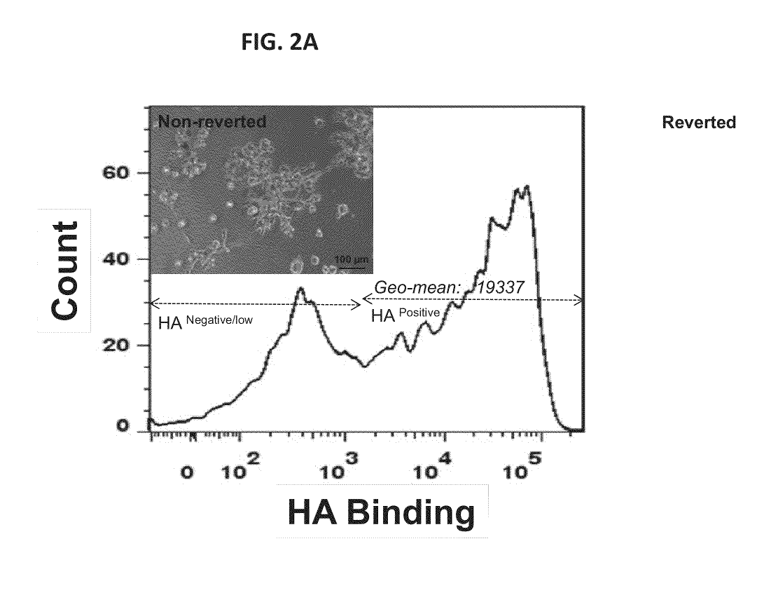

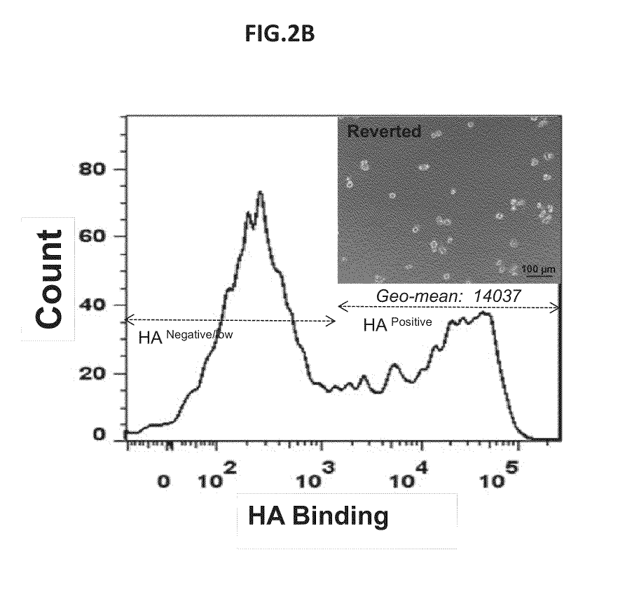

Multiplexed analysis of HA receptor display and HA binding in MDA-MB-231 cells showed that all cells that expressed cell-surface CD44 and RHAMM bound to HA probe (FIG. 1). Therefore, functionality of HA had not been altered by fluorescent modification, and the HA probe binding was related to the HA-receptor display.

We showed that HA uptake by MDA-MB-231 is inhibited by anti-CD44 antibodies using confocal imaging and that the signal of HA probe uptake is reduced by treatment with excess HA, added either during or prior to addition of HA probe. We have also assessed the relative importance of RHAMM vs. CD44 in HA uptake using mouse fibroblast cells that are RHAMM−/−, CD44−/−, and RHAMM−/−:CD44−/− originally isolated from RHAMM−/−, CD44−/− and double knockout mouse strains that were developed in Turley Lab.

Identification, Isolation and Characterization of HA-Targeted Subpopulations.

The heterogeneous HA binding profiles (non-Gaussian distribution) of the BCA cell lines permitted consistent identification and isolation of cell subpopulations that stably exhibited high and −/low HA binding (FIG. 3).

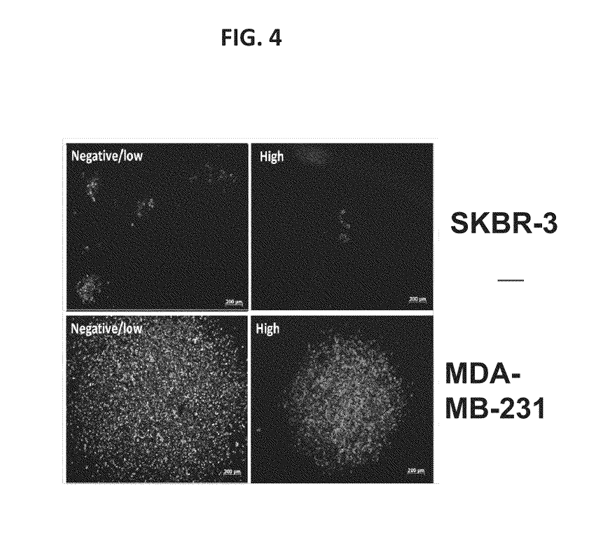

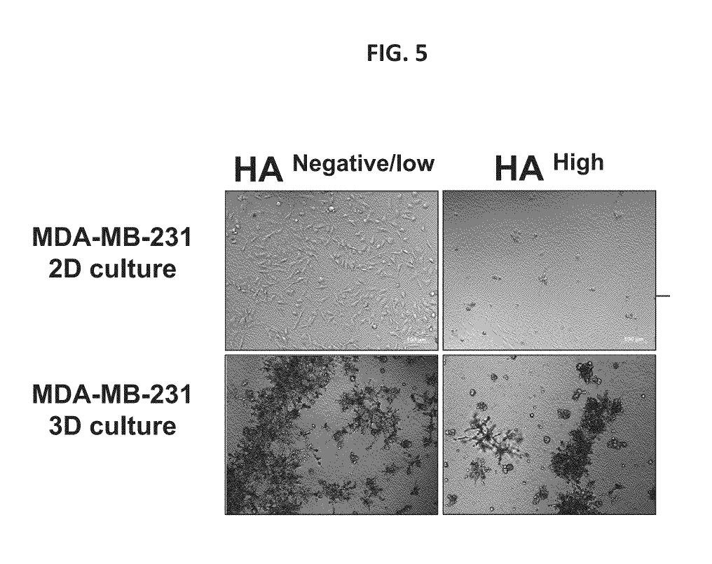

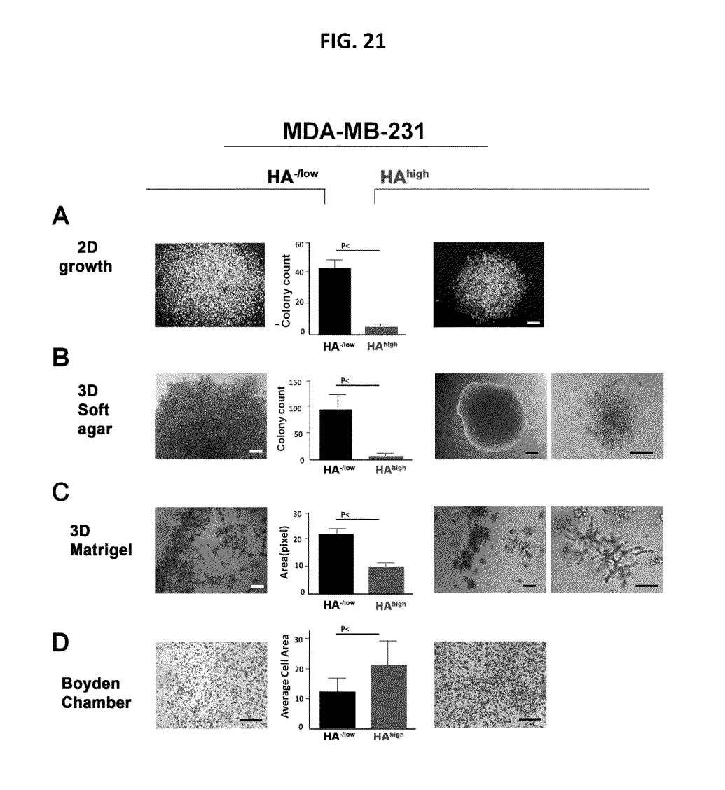

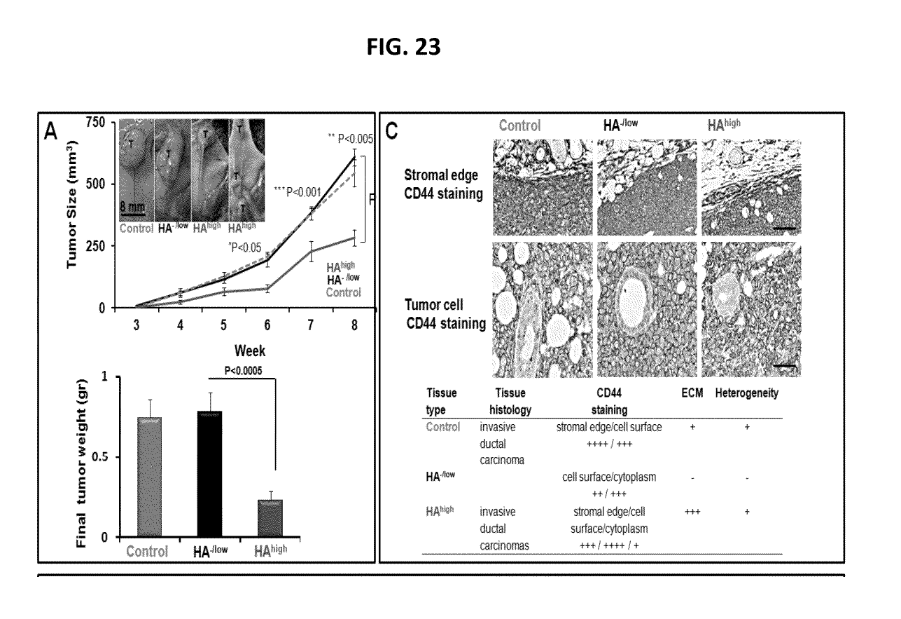

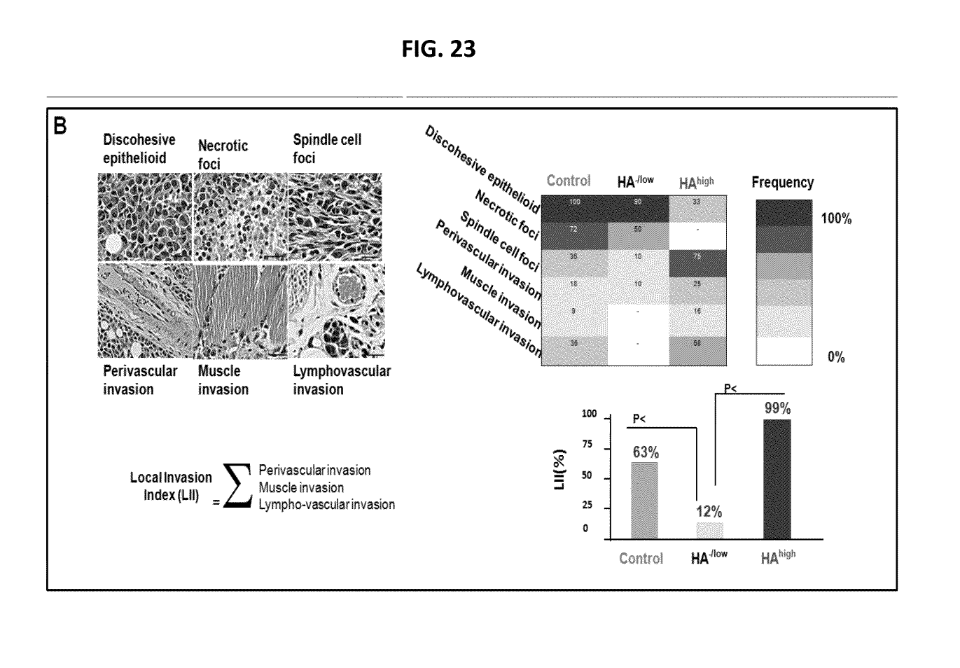

These two subpopulations exhibited different morphological phenotypes and proliferation rate. In culture, the HA^{High} cells (with surface phenotype of CD44⁺/CD24⁻/RHAMM in basal BCA lines and CD44^{-/low}/CD24⁺/RHAMM⁺⁺ in luminal BCA lines) were initially round, poorly adherent, and proliferated slowly but regained the morphological heterogeneity of the parent population with time (FIGS. 5 and 23). When cultured at a low density as single cells (colony-forming assays on 2D plastic), the HA^{-/low} subpopulation formed colonies by 13 days. The number and the size of HA^{-/low} colonies were higher than those formed by HA^{High} subpopulations (e.g. size ratio [HA^{-/low}/HA^{High}]: 4.21 and the count ratio [HA^{-/low}/HA^{High}]: 11.5 for MDA-MB231 cells). The growth differences between HA and HA subpopulations were also distinct in SKBR-3 cell line (FIG. 4), with HA^{-/low} subpopulation growing significantly faster. As such, in 3D culture on Matrigel, the HA^{-/low} subpopulation proliferated faster and formed uniform morphology (spread-branched for MDA-MB231 and spread-grape-like for SKBR-3). However, the HA^{High} subpopulation proliferated slowly and formed heterogeneous morphology composed of both spread-branched and compact-branched morphology (FIG. 5). Similar growth observations were made when the subpopulations were cultured in soft agar (FIG. 21).

We also established methods for high-resolution examination of internalization events and surface topographies of cells grown as 2D or 3D on MEMS platforms (plain or patterned substrates). FIG. 1C shows an example of topography evaluation in MDA-MB-231 cells. The HA-bound MDA-MB-231 cells formed nano-aggregates on their surfaces, which had larger sizes than the luminal cells (like MCF-7) that had low HA-binding capacity.

Evaluating Tumorigenicity of HA^{-/low} and HA^{High} BCA Subpopulations.

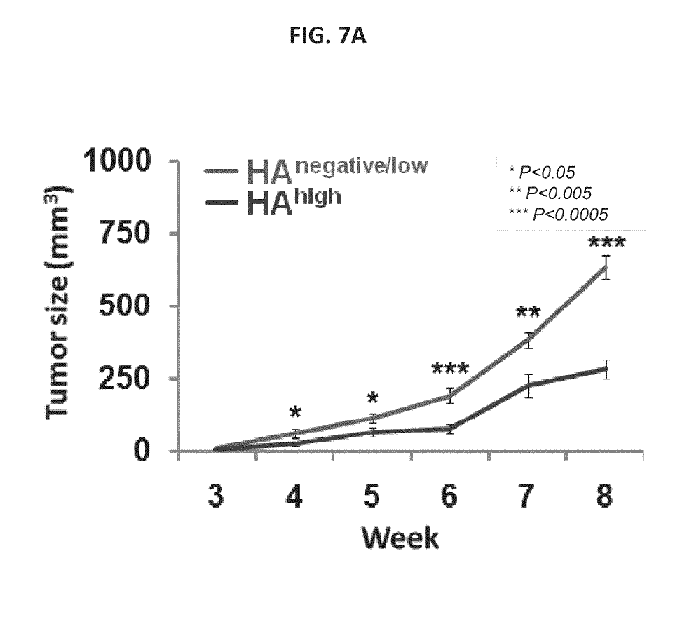

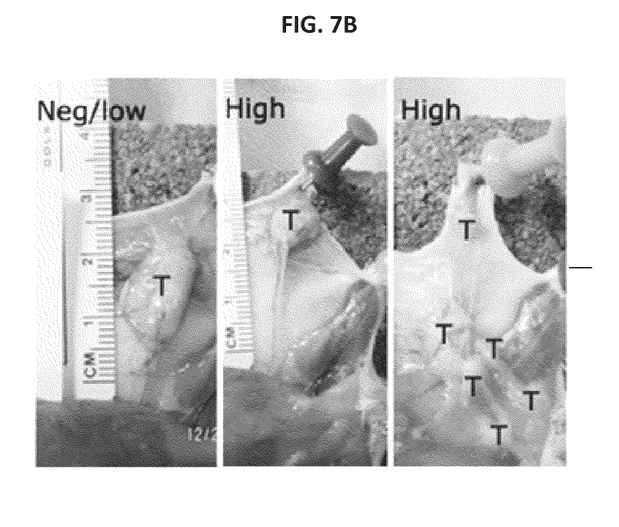

We characterized the tumorigenicity of HA^{High} and HA^{-/low} subpopulations subpopulations (FIGS. 7A and 23). MDA-MB-231 cells were injected as cell suspensions in Matrigel into the mammary fat pads of female NOD-SCID IL2−/− (severely immunocompromised). Animals were palpated weekly for tumor formation. Tumors were excised, weighed and processed for paraffin processed tissue sections. Evidence of local invasion and metastatic nodules in other tissues were also assessed visually (FIGS. 7B and 23).

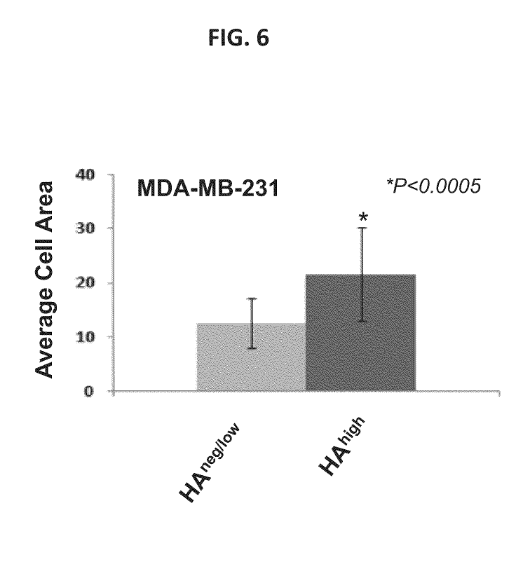

In agreement with the 2D and 3D proliferation observation, the tumorigenic index was significantly higher in the HA^{-/low} than in the HA^{High} subpopulations. Despite their lower tumorigenicity (e.g. tumor growth rate), we found that HA^{High} subpopulations often enriched for subsets that invaded aggressively into the mammary fat pad and peritoneum (gross morphology shown by multiple T markers, FIGS. 7B and 23). We became very interested in this observation and further examined the tumor phenotypes of these subpopulations. We observed that both subpopulations formed high grade adenocarcinoma. However, tumors formed from HA^{High} subpopulation were lymphovasculature positive, i.e., able to invade lymphovascualature, which is a pathological indicator of invasion in the clinic. In vitro analysis of both subpopulations in Matrigel-coated Boyden chambers indicated significantly higher motility for HA^{High} subpopulation, which confirmed our pathological observation (FIG. 6).

Assessment of HA-Based Probes for Imaging Tissues and their Microenvironments.

We evaluated the targeting ability of HA-based probes in several modalities (radiolabel, magnetic resonance, and fluorescence) to areas of microenvironment remodeling that are associated with both tissue injury and tumor formation. We chose well-characterized rat balloon injured carotid arteries as a response to injury model, and xenografts of MDA-MB-231 cells as a tumor model. Our results showed a selective targeting of HA to injured and tumor tissues regardless of the label. These results indicate that HA-based probes show promise as imaging agents that target to sites of remodeling microenvironments. As a key step for imaging probe development, we assessed whether HA-based imaging probes exhibited sufficiently long half lives following their intravenous (I.V.) injection and quantified the kinetics of HA elimination from plasma by analyzing data obtained from Hyal Corporation (Toronto, Calif.) as a phase I clinical trial on healthy human subjects and rats. HA injected I.V. (1-12 mg/kg) was well tolerated, and analysis of HA serum levels revealed a T½ of 12 hrs (for a 1.5 mg/kg dose), which was sufficiently long for use as imaging agents.

### Example 3

### Flow Cytometry Using Ha Metabolism

A biopsy of tissue can be obtained from a patient. Using flow cytometry, the cells can be sorted based on their ability to actively metabolize HA.

For example, a sample protocol using the Alexa Fluor 647 HA assay, then sorting using a flow cytometer, is described below:

(1) Grow cells to 50% confluence on culture flask; (2) Rinse in Ca²⁺-free Hank's Buffered Saline Solution (HBSS/20 mM HEPES, pH 7.3); (3) Harvest cells with non-enzymatic HBSS-based cell dissociation solution; (4) Resuspend in cold and sterile PBS solution that contains Antibiotic, HEPES pH 7.4, Insulin, and Transferrin (PBS/additive solution); (5) Centrifuge and eliminate supernatant; (6) Wash in cold PBS/additive solution; (7) Count cells using cell counter instrument and measure viability; (8) Treat cells (in suspension) with Alexa Flour 647-HA; (9) Incubate for 45 min on ice, in dark.

For single-color HA-binding flow cytometry: (a) wash the cells, eliminate supernatant and adjust concentration of solution appropriate for flow cytometry and (b) perform live cell flow cytometry immediately.

For multiplexed analysis: (a) wash the cells and eliminate supernatant; (b) block in cold FACS buffer solution (FCS/HBSS/HEPES) solution for about 40 minutes on ice, in dark; (c) wash the cells with PBS/BSA solution to remove excess FACS buffer and adjust concentration appropriate for antibody staining; (d) incubate cells with relevant concentrations of each antibody (Anti-CD44, Anti-RHAMM, and Anti-CD24) for about 45 min on ice, in dark; (e) wash the cells, eliminate supernatant and adjust concentration of solution appropriate for flow cytometry; and (f) perform live cell multi-color flow cytometry immediately.

In fact, this method of sorting by HA metabolism would be extremely useful for identifying the very invasive population in leukemia; clinicians often use flow cytometry and FACS on actual patient samples to phenotype cells. If you are selected as a patient to receive stem cell therapy for Hodgkins lymphoma, they sort through your existing stem cell in bone marrow as well as peripheral stem cells. This method would really allow them to see the invasive subsets that might not be detected otherwise. They have a very crude way to decide whether you go through a stem cell transplant, and without it you only live about 5 years. If your tumor is not below your diaphragm, they assume that it has not spread into your bone marrow only by correlation and will give you a stem cell transplant. Furthermore, tumor size is the main prediction of whether it is good or bad tumor. If they have a method by going through the cells that are in your blood which further taking the stem cells from and exclude those from the ones that bind to a large amount of HA and are possibly tumorigenic and very invasive, that would improve the outcome.

### Example 4

### Flow Cytometry Using HA Metabolism

For this study, we chose to examine and relate HA:BCa binding to the cell-surface CD44 and RHAMM/HMMR because they bind to different ranges of HA sizes and are implicated in BCA progression [Sironen, Tammi et al. 2011; Veiseh and Turley 2011; Wang, de la Motte et al. 2011; Neri and Bahlis 2012; Turley and Naor 2012) (Gao, Liu et al. 2010; Jiang, Liang et al. 2011; Tolg, Hamilton et al. 2012). For example, The HA receptors CD44 and RHAMM are commonly elevated in BCA and associated with poor clinical outcome. Elevated CD44 protein expression in breast carcinoma cells is related to poor differentiation, postmenopausal status and triple negative BCA. Elevated CD44 protein expression in the peri-tumor stroma is associated with HER2+ BCA increased incidence of relapse and shortened overall survival (Auvinen, Tammi et al. 2012). Elevated RHAMM expression in tumor subsets is a prognostic indicator for poor outcome and increased perhipheral metastasis in BCA (Wang, Thor et al. 1998) (Maxwell, McCarthy et al. 2008; Turley and Naor 2012) and as a result of its links to BRCA1 may also be a factor in BCA susceptibility (Maxwell et al 2012).

We compared the binding of polydisperse, fluorescent HA (MW range, 10-480 kDa, 240 kDa MWav) to BCa lines of different molecular subtypes and relate binding/uptake heterogeneity to the display of CD44 and RHAMM/HMMR. We show that HA binding is heterogeneous and this property may be related to heterogeneity of RHAMM/HMMR display resulting from exposure to HA. BCa lines sorted according to levels of fluorescent HA binding display quite different phenotypes. Those that bind high levels of HA are highly invasive but poorly proliferative while those that bind low levels of HA are poorly invasive and highly proliferative. We conclude that monitoring HA binding profiles in BCa lines has revealed a novel form of tumor heterogeneity that predicts malignant behavior. Our results further suggest that HA/RHAMM interactions may a target for both detection of aggressive BCA tumors and therapeutic intervention in these and other cancers.

### Materials and Methods

Two-Dimensional (2D) Culture:

Cells were grown to 50%-70% confluence in 75 cm² culture flasks in DMEM supplemented with 10% FCS at 37° C. and 5% CO₂, then subcultured every 3-4 days. For HA uptake experiments, cells were grown to 50% subconfluence, which was achieved 12-20 hrs after subculture, in order to maximize RHAMM surface display. Cells were rinsed in Ca++-free Hanks Buffered Saline (HBSS) solution/20 mM HEPES, pH 7.3, and removed from the substratum by incubating in non-enzymatic HBSS-based solution (Sigma-Aldrich[H-6648]) for 2-3 min, which preserves cell surface epitopes (Veiseh M, et al, Biomacromolecules 2012, Hamilton S. R. et al, JBC, 2007). Viablity of cells was assessed using a Vi_CELL cell analyzer.

Three-Dimensional (3D) Culture:

Cells were prepared for culturing “on top” of matrigel according to Lee et. al (Lee G., et al, Nature Methods, 2007). Culture of post sorted BCa cells required minor modifications of this method. Briefly, for unsorted parental BCa cells, tissue culture dishes were placed on ice prior to coating with ice-cold Matrigel (BD Biosciences, 160-200 μl coat for 1.8 cm² surface area) then incubated at 37° C. for 15-20 min to promote polymerization of the matrigel. Viable cells that were removed from 2D tissue culture surface using 0.05% Trypsin (Sigma) were suspended in ice-cold culture medium (20,000 cells/250 μl for 1.8 cm² surface area) and added drop wise to the matrigel-coated wells at RT. After settling for 2-5 min under sterile hood to promote cell attachment, a 10% solution of matrigel in ice-cold medium (25 μl/250 μl) was added to the cells to make a final concentration of 5% Matrigel. Cells were maintained in incubators (37° C. and 5% CO₂) and culture medium was changed on alternate days. FACS-sorted BCa cells were cultured in a similar manner except that the suspended cells in ice-cold medium (20,000 cells/250 μl for 1.8 cm² surface area) were mixed with 10% solution of ice-cold Matrigel at equal medium volume (250 μl) and were added drop-by-drop to the surface of matrigel coated wells, prepared as above. Cells were then incubated and (500 μl) culture medium was added every two days.

3D Culture “Reversion” Assay.

MDA-MB-231 cells were cultured in 3D as described above for unsorted cells. At the time of culture, a function blocking anti-β1 integrin antibody (AIIB2, Aragen Bioscience) was added (10 μg/ml) to cell suspension and the mixture was seeded following “on top” procedure described above. On the second day of 3D culture, 2 mM LY294002 (Calbiochem), a small molecule inhibitor of PI3K was added to the medium. At day 4, reverted (AIIB2+LY294002 treated) and non-reverted (control untreated cells) were photographed with a Zeiss Axiovert microscope prior to releasing cells from their matrices using PBS-EDTA as described below for use as single cells in subsequent assays such as A⁶⁴⁷-HA binding/FACS.

Extraction of Cells from 3D Cultures.

PBS-ethylenediaminetetraacetic acid PBS-EDTA solution (5 mM EDTA Invitrogen, 1 mM NaVO4, 1.5 mM NaF, 100× protease inhibitor cocktail in 1×PBS) was prepared, filtered, and placed on ice along with cell culture plates and conical tubes that were coated with 5% BSA solution 1×PBS (Sigma-Aldrich) in for 15 minutes at RT prior to this stage. 3D cultures were washed with 1 volume culture medium followed by 2 washes with 2 volumes of PBS-EDTA. Matrigels were detached from bottom of culture wells using polyethylene cell scrapers (Corning, Inc.), and collected into ice-cold 15 ml conical tubes. Conical tubes containing the polymerized matrigel and cells were oriented horizontally on an ice bucket, which was shaken on a Thermoscientific shaker at 4° C. for 1 hr to de-polymerize and liquify the Matrigel. Tubes were then centrifuged at 800 rpm for 5 min, the supernatant was discarded, and cells were resuspended in the PBS-EDTA solution. This was repeated 2× to minimize the amount of Matrigel in the final extract and cells were counted using Vi CELL cell analyzer.

HA Probe Synthesis.

0.2 ml of soluble HA (1% HA, MWav=280,000 dalton) was activated in 1 ml of a (0.0028 g/1 ml MES) N-(3-Dimethylaminopropyl)-N′-ethylcarbodiimide hydrochloride (EDAC) solution (Sigma-Aldrich) in 4-morpholineethanesulfonic acid (MES) (20 mM, pH 4.5, Sigma-Aldrich), and the resulting solution was reacted with 0.3 ml of A⁶⁴⁷-HA hydrazide, tris(triethylammonium) salt (Invitrogen, 1 mg/ml in H₂O) in the dark at RT. The solution was covered in aluminum foil and placed on a rocker for 12 hrs at room temperature. Unconjugated dye was removed by dialysis against 75 mM NaCl in PBS (8.8 g NaCl in 2L of 1×PBS) using a Slide-A-Lyzer 10K MWCO dialysis cassette (3-12 ml capacity, Pierce [66380]) in the dark for up to 4 days at 4° C. NaCl/PBS was changed four times and the solution retained in cassettes (A⁶⁴⁷-HA) was sterilized by filtering through a 0.2 μm filter.

RHAMM Antibody Preparation.

Mouse monoclonal anti-HMMR prepared against a purified recombinant human HMMR peptide representing aa706-767 (including the HA-binding region from aa719-750; clone 6B7D8) (ProMab; 1:900) was incubated for 60 min at room temperature followed by rinsing with TBST and detected using a streptavadin-horseradish peroxidase detection kit (Covance, Princeton, N.J.) using the manufacturer's specifications. All slides were then rinsed in TBST followed by detection using diaminobenzidine (DAB) (Covance). Slides were incubated for 5 min followed by rinsing with TBS, counterstaining with CAT Hematoxylin (Biocare) for 5 min, dehydrating, and coverslipping.

Multi Color Sample Preparation for Flow Cytometry:

We established protocols for minimum decoration of cells by fluorescent HA and antibodies, maximum detection sensitivity at the cell surface, and retained viability/functionality during and after sorting. Three fluorochromes were selected to minimize fluorescent spectral overlaps and to maximize brightness for low-expressing markers (i.e. RHAMM) providing sensitive monitoring of subtle changes in marker expression and HA binding capacity in live cells. The far red/near infrared fluorescence range was selected for HA to extend signal stability required for the multiple stages of characterization during these experiments (e.g. A⁶⁴⁷-HA). Multicolor staining was performed on cells that had been incubated with A⁶⁴⁷-HA as described above. Prior to addition of antibodies cells were blocked in a cold HBSS solution (5% FCS+HBSS/20 mM HEPES) for 45 min in dark then washed twice in a PBS/2% BSA solution (Sigma-Aldrich). Aliquots of 1.25×10⁵ cells/50 μL were incubated with anti-RHAMM-A⁴⁸⁸-HA 6B7B7 monoclonal antibody (2.5 μL) and rat anti-mouse CD44−PE Cy™5 (0.75 μL of 0.2 mg/ml) simultaneously, on ice for 30 min in the dark. Cells were then washed 2× in cold 5% FCS/HBSS/HEPES buffer to remove unbound primary antibodies, filtered through cell strainers, reacted with PI (as needed) and analyze, as described above.

Flow Cytometry Parameters for Single Color Analysis:

Forward light scatter (FSC) was collected through a neutral density filter in the forward light scatter path, and side scatter (SSC) was collected through a neutral density filter at a 90-degree angle. The 488 nm lasers excited A⁴⁸⁸-HA and PE, while the 633 nm laser excited A⁶⁴⁷-HA and APC. Fluorescence emissions were collected though the FITC (533/30 BP), PE (585/42 BP), and APC (660/20 BP) filters in fluorescence channels FL1, FL2 and FL4, respectively. Unstained cells were first run as controls, and 30,000 events were analyzed for each sample. FSC and SSC settings were adjusted until the cells appeared in the middle of the FSC vs. SSC dot plot so that any cell aggregates or debris was excluded. The PMT voltage of the FITC, APC and PE detectors was adjusted until the cells appeared within the lower quadrant of the different dot plot, setting the background or unstained fluorescence levels according to these parameters. Next, single color samples were analyzed as needed. To verify antibody specificity, we ran the positive control and the isotyped-matched antibody for every single color sample (PI, RHAMM, CD24, CD44, HA).

Flow Cyotmetry Parameters for Multiplexed Analysis:

With the above parameters set, single-color samples were run, and the spectral overlaps were corrected using digital compensation by subtracting the overlap signal of single colors from the overall signals detected in each channel. Compensation was applied to confirm the positive population was directly horizontal or vertical to the negative population and not detectable in the other detector regions. HIC compensation was not required for the APC detector but was checked. The initial fluorescent levels of control unlabeled or non-immune IgG treated cells in the absence of added HA was measured for the RHAMM (FL-1) and CD44 (FL-4) channels and compared to the real-time display of RHAMM and CD44 (detected by antibodies) as well as A⁶⁴⁷-HA binding. Compensation was performed using single color samples as above and for double and triple color samples (RHAMM/CD44 and PI/RHAMM/HA) with spectral overlap. After these parameters for defining background/negative signal and compensation values were established, the multiplexed three-color samples (RHAMM/CD44/HA) were analyzed.

Fluorescence Activated Cell Sorting:

A⁶⁴⁷-HA bound cells were sorted using gates set to an 8% threshold on a histogram profile. The threshold was set based upon the intensity with which cells bound A⁶⁴⁷-HA and were sorted into −/low and high subpopulations (designated as HA^{-/low} and HA^{high} subpopulations). Sorted cells were collected in fresh culture medium and cultured under 2D or 3D conditions as described above.

Cell morphology and growth analyses.

Immediately Following Sorting by FACS, HA^{-/low} and HA^{high} subpopulations of MDA-MB-231 were plated at equal densities in triplicates onto 4 well glass chamber slides (10,000 cells/well) or onto matrigel as described above. Cells were photographed on alternate days for 8 days using a xx microscope to evaluate cell morphology. The occupied surface areas by grown cells were quantified by particle analyzer module of ImageJ software. For proliferation evaluation, the HA^{-/low} and HA^{high} subpopulations were plated in 24-well plates in DMEM plus 10% FBS and incubated at 37° C. At each time point (1, 3, 5, 7 days) they were dissociated with 0.05% trypsin and counted by Vi_CELL cell analyzer.

Colony Forming Assays.

Immediately following sorting by FACS, HA^{-/low} and HA^{high} subpopulations of MDA-MB-231 cells were evaluated for their colony-forming ability in 2D culture and in soft agar. 2D culture: Single-cell suspensions were seeded into 6-well plates (1×10³ cells/well) in DMEM plus 10% FBS and incubated at 37° C. for 13 days. Cells were stained with 0.2% methylene blue in 50% ethanol and destained with tap water. Each well was photographed entirely, and the number of colonies was counted. 3D: 1% agar in H₂O was mixed with equal volumes of 2×DMEM plus 20% FCS and 2% antibiotic at 40° C. 1 ml of this solution (base agar) was poured into a 35-mm plate in triplicate and solidified at RT. 0.7% agar solution equilibrated to 40° C. was mixed with 2×DMEM and the sorted subpopulations at 5,000 cells/ml, and poured onto the base agar at 1 ml/plate (4 plates per condition). After solidification of the top agar/cell layer at room temperature 500 μl of the base agar solution was added to each plate and they were placed at 37° C. incubator for 1 hr. The solidified multilayered agar plates were covered with 500 μl of DMEM containing 10% FCS and 1% antibiotic and were incubated in a humidified atmosphere containing 5% CO₂ at 37° C. for 2 months. Fresh culture medium was added every other day. Plates were plates were photographed on a Zeiss Axiovert 200 microscope prior to staining with 0.01% crystal violet for 30 minutes, and colonies were counted.

Cell Cycle Assessment.

HA^{-/low} and HA^{high} subpopulations of MDA-MB-231 cells (˜350,000 cells) were collected into fresh medium, washed (2×) with HBSS, and fixed by ice-cold 70% ethanol in PBS which was added drop-by-drop to a final volume of 1 ml. Fixed cells were stored at 4° C. for at least 24 hrs before fixative was aspirated. Cell pellet was resuspended in the 25 μl supernatant and were exposed to 105 μl RNase A of 0.6 mg/ml in HBSS for 30 min at RT to remove RNA from the solution. Cells were washed by 500 μl HBSS solution and centrifugation to remove RNA. Cell pellet was dissolved in 350 μl HBSS followed by addition of propidium iodide (20 μg/ml) and filtration through cell strainers to dissociate cell aggregates. Samples were stored at 4° C. in the dark until FACS analysis was performed to detect amount of DNA in cells. Similar procedure was used for cells that grew as 2D monolayer after sort. Briefly, they were harvested from approximately 70% confluent plates by addition of 0.05% trypsin and were centrifuged at 800 rpm for 5 min. An aliquot of cells (1×10⁶ cells) in HBSS was fixed in ice-cold 70% ethanol and processed as described above.

Matrigel Invasion Assay.

Sorted cells were cultured in 2D T75 tissue culture flasks to expand cell number. Cells were serum starved (DMEM+0.2% FCS) overnight prior to use in invasion assays. Matrigel invasion chambers (BD Biosciences) were incubated with 500 μl serum-free media for 2 hrs at 37° C. Cells were washed with HBSS, dissociated by non-enzymatic HBSS-based cell dissociation medium, centrifuged to remove dissociation medium, resuspended in DMEM+0.2% FCS, and counted by Vi_CELL cell analyzer. The medium in the matrigel invasion chambers was removed and replaced with 500 μl DMEM+10% FCS. 5×10⁴ cells suspended in DMEM+0.2% FCS were added into each chamber, and then DMEM+0.2% FCS was added to each chamber to achieve a final volume of 300 μl. Chambers were incubated at 37° C. for 1 day. Cells that had migrated/invaded across the filters into the lower invasion chamber were stained with 0.125% Coomassie Blue in methanol:acetic acid:H2O (45:10:45, v/v/v) for 20 min. Inserts were then rinsed with water, air-dried for 20 minutes and photographed. Invaded cells were quantified as cell area using particle analyzer module of ImageJ.

Tumor Xenografts in Severely Immune-Compromised Mice:

Female NOD-SCID IL2^{-/-} (NOD.Cg-Prkdc^{scid}Il2rg^{tm1Wj1/SzJ}) mice were purchased from the Jackson Lab (005557) and housed according to animal ethics requirements at five per cage with unlimited chow and water in a controlled animal barrier (AWRC 26R-0529-1303). Six to twelve mice were randomly chosen and assigned to each of the following three groups: #1=unsorted MDA-MB-231 cells; #2=sorted HA^{-/low} MDA-MB-231 subpopulation; and #3=sorted HA^{high} MDA-MB-231 subpopulation. 62.5×10³ cells were suspended in 20 μL of Matrigel/DMEM (10% FCS) (1:1 vol) on ice then injected into the left fourth mammary fat pad of anesthetized mice under isofluorane gas. Mice were maintained under aseptic sterile conditions, and received antibiotics in their drinking water for 2 weeks post-surgery. Tumor size was monitored using vernier calipers for 3-8 weeks and this value was used to calculate the tumor volume [L×W²×0.5 (where 1 cm³=1 g)]. At 8 weeks, animals were euthanized in CO₂ chambers and tumors were harvested, weighed to obtain wet tumor weight and fixed in 2% paraformaldehyde in PBS.

Immunohistochemistry:

Fixed tissue was paraffin processed and histology sections were prepared at the Mouse Pathology Core of the Helen Diller Family Comprehensive Cancer Center (University of California, San Francisco). CD44 was detected in de-paraffinized sections using a rabbit monoclonal antibody (Millipore, anti-CD44 antibody clone EPR1013Y) according to manufacturer's instructions. Bound anti-CD44 antibody was detected with anti-Rabbit-HRP (Sigma). Non-immune rabbit IgG was used as a control to establish the specificity of anti-CD44 antibody staining Stained slides were scanned and quantified for amount of staining using Image J.

Quantitation of Extravasation Efficiency Rates in Chicken Embryos:

Fertilized chicken eggs were incubated in a rotary incubator at 37° C. with 90% humidity for 4 days before being removed from the shell and placed in covered dishes and incubated at 37° C. with 90% humidity until usage (PMID:20671724). On day 13 of embryonic development, 7.5×10⁴ tumor cells in 100 μL PBS were intravenously injected into a vein within the chorioallantoic membrane (CAM, N=5 each group) (PMID-20671724). Prior to injection, cancer cells were pre-treated with HA (100 mg/ml) or vehicle (PBS) for 1 hour on ice. Immediately after injection, four aluminum foil square markers (5 mm diameter) were placed on the surface of the CAM to form a large rectangular region of interest (ROI) for macroscopic imaging. Stitched images of the ROI were acquired on an upright Zeiss Examiner and a Hamamatsu EMCDD camera using Volocity software (Perkin Elmer Inc.). Stitched images of each animal's ROI was taken immediately after injection and at 24 hours post-injection. To confirm successful extravasation, CAM vasculature was labeled with fluorescent Lens Culinaris Agglutinin (LCA, Vector Laboratories, Burlingame, Calif., USA) at 24 hrs post-injection timepoint and cancer cells were visualized using a spinning-disk confocal microscope (Quorum Technologies, Waterloo, ON, Canada)(PMID: 21765460). Intravascular cells were identified as being present\ only within the CAM lumen as labeled by LCA. Extravascular cells were identified as being present within the underlying stroma, and not within the same Z-plane as the CAM lumen (PMID: 21765460). At least 200 cells for each ROI at T=0 was analyzed and enumerated.

Uptake of A⁶⁴⁷-HA into MDA-MB-231 Tumor Cells in CAMs:

Unsorted MDA-MB-231 cells were grown to 50-70% sub-confluence in 2D culture flasks and then released from the substratum with non-enzymatic dissociation medium (Sigma). Released cells were washed in DMEM+10% FCS, resuspended in 1 ml DMEM then counted with a hemacytometer. 7.5×10⁴ tumor cells were incubated with 100 μl A⁶⁴⁷-HA in PBS (xx concentration) then immediately injected into a vein in the chorioallantoic membrane of day 13 chick embryos as above. Timelapse confocal images of tumor cells that were arrested within the vein were taken 30 min after injection.

Assessment of Deviation from Normality.

Deviations from the normal distribution were analyzed by Shapiro-Wilk normality test (Shapiro, S. S.; Wilk, M. B., 1965) of (Prism 5.0 software, GraphPad Inc, San Diego, Calif.) after log transformation. The test statistic is:

$$W = \frac{{( {\sum }_{i = 1}^{n} {a}_{i} {x}_{( i )} )}^{2}}{{\sum }_{i = 1}^{n} {( {x}_{i} - \overline{x} )}^{2}}$$

where x₍ᵢ₎ (with parentheses enclosing the subscript index i) is the ith order statistic, i.e., the ith-smallest number in the sample and x̅=(x₁+ . . . +xₙ)/n is the sample mean; the constants aᵢ are given by

where

$$( {a}_{1} , … , {a}_{n} ) = \frac{{m}^{T} {V}^{- 1}}{{( {m}^{T} {V}^{- 1} {V}^{- 1} m )}^{1 / 2}}$$

$$where$$

$$m = {( {m}_{1} , … , {m}_{n} )}^{T}$$

and m_{—}1, m_n are the expected values of the order statistics of independent and identically distributed random variables sampled from the standard normal distribution, and V is the covariance matrix of those order statistics.

CD44 and RHAMM Co-Expression in BCa and BCa Lines of Different Subtypes.

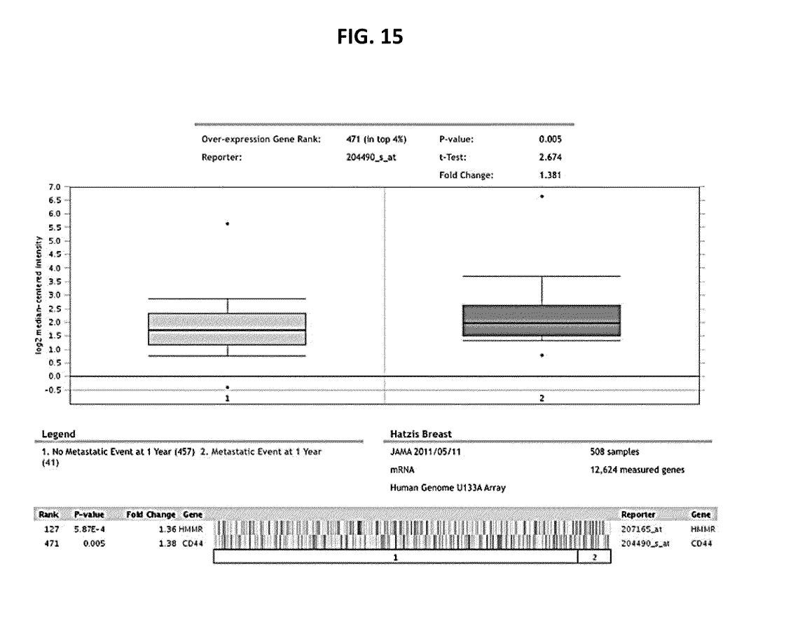

Most human BCA lines have been grouped into subtypes based upon their molecular signatures (Kristensen, V. N. et al. Proc Natl Acad Sci USA, 2011; Sørlie, T. et al. Proc Natl Acad Sci USA 2001; and Perou, C. M. et al. Nature 406, 747-752., 2000) but few of these have been characterized for CD44 or RHAMM expression (Sheridan, Kishimoto et al. 2006; Hamilton, Fard et al. 2007; Veiseh, Breadner et al. 2012) and to our knowledge none have been examined for HA binding. Assuming that HA receptor display is proportional to HA binding, we utilized knowledge based data banks such as Oncomine to identify the BCa subtypes that express highest levels of RHAMM and CD44, thus allowing us to select appropriate BCa cell lines as clinically relevant models. These data banks also allowed us to the types of BCa tumors, which co-express RHAMM and CD44 and which are linked to poor clinical outcome. These analyses showed that CD44 mRNA expression is upregulated relative to normal breast tissue in triple negative (basal) but not luminal tumors and that CD44/RHAMM co-expression in this subtype is linked to poor outcome in 80% of CD44+ve triple negative tumors (FIG. 15). In contrast, RHAMM expression is elevated relative to normal breast tissue across subtypes (12/13 data sets) and is often linked by itself to poor outcome (e.g. 18/27 data sets). We therefore focused upon basal (triple negative ER−, PR−, Her2−) and luminal (ER+, PR+, Her2− and ER−, PR−, Her2+) BCa line subtypes for our analyses and chose 9 BCa lines for initial analyses: MDA-MB-231 (basal) SUM1315MO2 (basal), HS-578T (basal), T4 (basal), HCC-1569 (luminal), MCF-7 (luminal), SKBR-3 (luminal), MDA-MB-361 (basal) and BT474 (luminal). We first confirmed that each of these lines display CD44 and/or RHAMM (Table 5).

As predicted from Oncomine data analysis of mRNA expression, CD44 display was high in triple negative BCa and low or absent in luminal BCa lines. In contrast, RHAMM was displayed in all BCa line subtypes examined, although levels were higher in luminal than in triple negative lines. We expected that since all these lines expressed HA receptors, they would also bind to HA, and further that lines expressing both RHAMM and CD44 would bind more HA. To assess this, we synthesized fluorescent HA (F-HA) (FIG. 15A), added this to these basal and luminal BCa cell lines and quantified the binding levels using flow cytometry (Table 5). Indeed, all 9 BCa lines bound F-HA although in varying amounts. In general and consistent with our assumptions, BCa lines that express both CD44 and RHAMM bind greater amounts of F-HA binding and this property is most typically exhibited by triple negative subtypes (Table 1, 4, and 5). Based upon these results we selected MDA-MB-231/T4-2 and MCF-7/SKBR-3 as examples of triple negative and luminal subtypes, respectively, for further analyses of the relationship amongst HA binding levels, binding heterogeneity in subpopulations and tumorigenesis.

HA Binding to BCa Cell Lines is Heterogeneous and Correlates with the Malignant Phenotype.

We first compared the cell population distribution of F-HA binding in these cell lines. We observed that F-HA binding is typically non-Gaussian in distribution (FIG. 16) suggesting that F-HA binding is heterogeneous. For example all F-HA binding profiles contained positively skewed tails (above 10³ binding signal) indicating the presence of small subpopulations of cells with very high levels of F-HA binding (FIG. 16B, +F-HA). Indeed, a Shapiro-Wilk normality test confirmed that all four F-HA binding profiles were significantly deviated from Gaussian distribution as measured by “W” (MDA-MB-231: 0.8528, T4-2: 0.7245, MCF-7: 0.6505, SKBR-3: 0.6265, P<0.001). However, the degree of tailing varied with BCa line subtype. For instance, basal MDA-MB-231 and T4-2 cells displayed more prominent tails than luminal MCF-7 or SKBR-3 BCa cells indicating that these basal lines contained greater numbers of cells with very high F-HA binding levels than the luminal counterparts. This difference in distribution of F-HA binding largely accounted for the higher geometric mean of fluorescent intensities observed in basal vs. luminal BCa line subtypes (Table 6). To quantify this property, we defined a heterogeneity index (Het. I), which takes into account the surface areas under curves (SUC) of the populations with HA binding intensities below and above control baseline value of 10³ (SUC1 and 2, respectively) as follows:

(Het.I.)=(SUC2/SUC1)

Comparison of Het.I. values among BCa lines showed the highest heterogeneity value for MDA-MB 231(1.078897218) followed by T4, SKBR-3, and MCF-7 (0.215870743, 0.213173792, 0.090063956). The rank order for HA binding heterogeneity and level were not time dependent; they were detected as early as 2 minutes after HA binding and remained robust over time (e.g. MDA-MB-231 vs. SKBR-3, FIG. 16 C).

To observe whether differences in heterogeneity and binding level would persist after cultivation in more physiological conditions, we grew cells on laminin rich gels (lrECM) in three dimensional (3D) cultures similar to tumor microenvironments in vivo, and subsequently performed HA binding analyses on cells in suspension after their release from the matrix gels. These culture conditions did not change the rank order of HA binding levels (e.g. MDA-MB-231 vs. SKBR-3, FIG. 16D) and the HA binding profiles remained non-Gaussian (e.g. FIG. 16E). These results indicate that F-HA binding characteristics of these cells is stable in both 2D and 3D culture environments.

To assess if F-HA binding levels and heterogeneity are related to a tumorigenic phenotype, we took advantage of our ability to ‘revert’ the malignant phenotype to a non-tumorigenic state that can be detected in 3D cultures. In this model, β1 integrin signaling was blunted with a function blocking anti-β1 integrin antibody combined with a PI3 kinase or MAP kinase inhibitor and allowed us to probe the relationship of HA binding levels and binding heterogeneity to tumorigenesis without altering the mutant genotype/subtype or genomic stability of the target cell line. The profiles of reverted tumor cells remained non-Gaussian and still exhibited considerable binding heterogeneity (compare FIGS. 16E and F). However, the percentage of cells with high F-HA intensity appeared to be selectively reduced (1.4 fold on a log scale) and those with the lowest F-HA were increased. These results suggest that the presence of subpopulations, which bind high levels of F-HA is linked to tumorigenicity.

HA Receptor Display and F-HA Binding Heterogeneity Differ in BCa Lines.

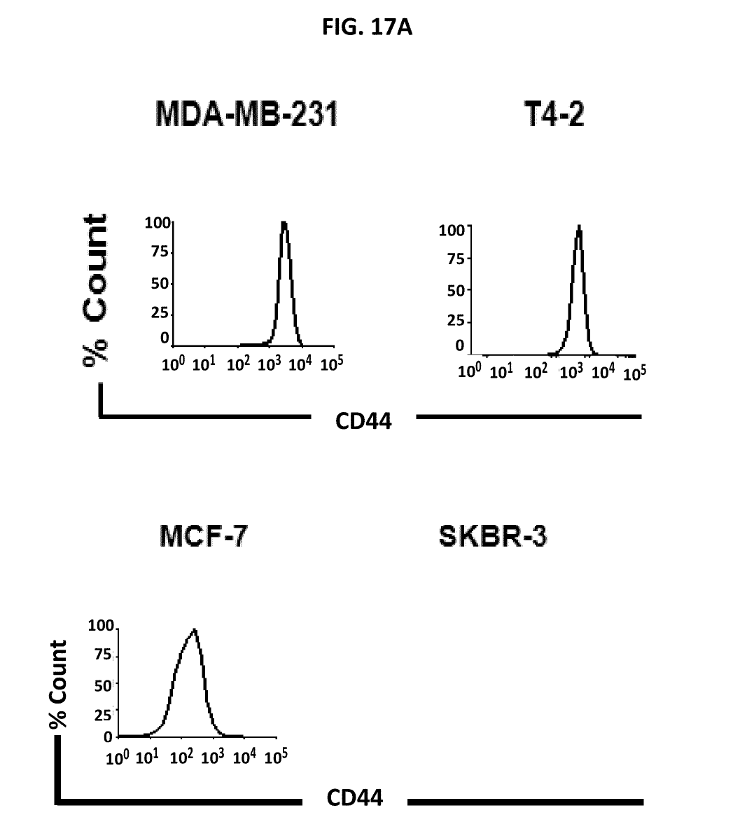

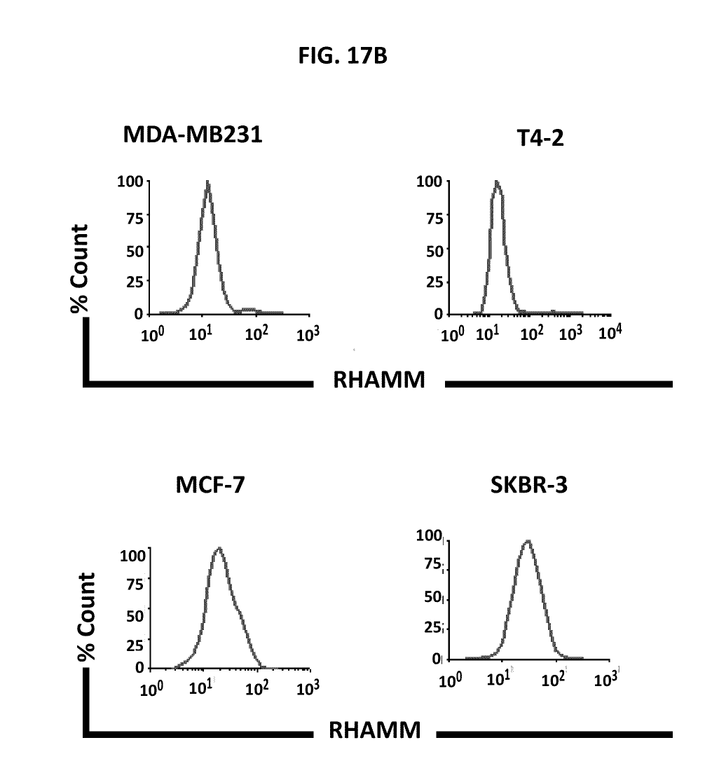

We next determined if the profiles or levels of CD44 and RHAMM display might account for F-HA binding heterogeneity. Flow cytometry profiles of both receptors displayed on either basal or luminal lines failed to have positively skewed tail as were observed for F-HA binding (FIG. 17). These results indicate that there is only a limited heterogeneity of HA receptor display in BCa lines regardless of subtype, and that there isn't a linear relationship between HA receptor display and F-HA binding, which accounts for the heterogeneity of F-HA binding. Previous studies have shown that the ability of some HA receptors, in particular CD44, to bind to HA is not constitutive but has to be activated through signaling cascades such as PKC (ref). Since HA activates PKC controlled pathways and RHAMM can mediate this effect, we next determined if the addition of HA to cells changed the HA receptor profile to more closely match the F-HA binding profiles.

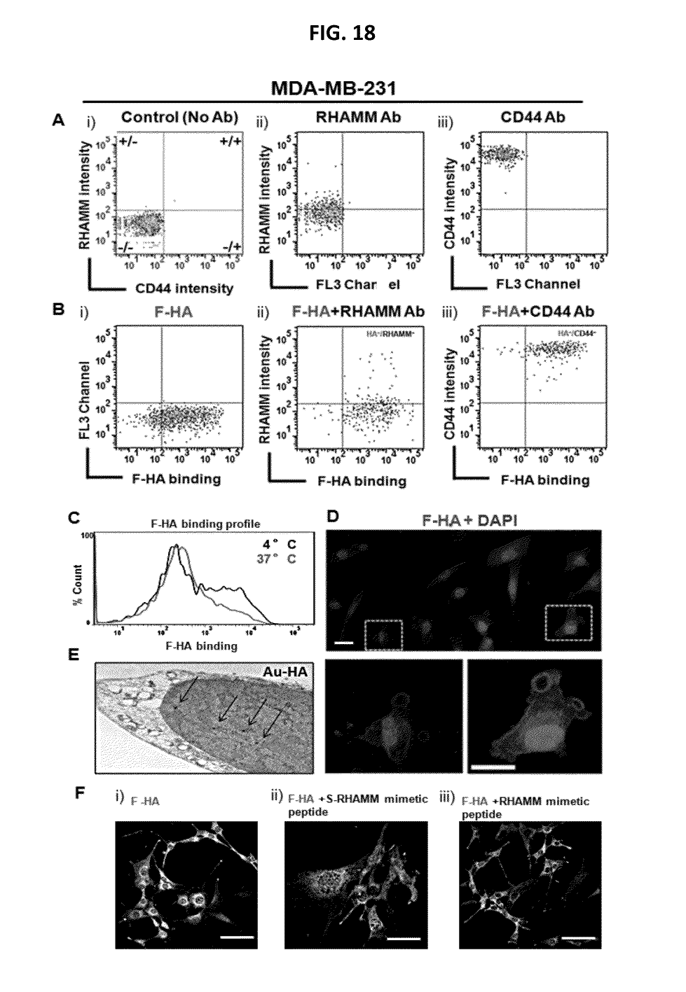

We therefore developed a multiplexed profiling strategy to detect the real-time display of CD44/RHAMM receptors and simultaneous binding of F-HA to these cells using flow cytometry. For these experiments, we utilized MDA-MB-231 cell line because it exhibits the highest HA binding and heterogeneity, expresses both CD44 and RHAMM at the surface, and is used by many investigators as a prototype of highly invasive triple-negative breast cancer. We quantified receptor display and F-HA binding levels as well as heterogeneity from these flow profiles using the Het-I described earlier to detect subtle changes in HA receptor display resulting from exposure to F-HA. The fluorescence characteristics of the cells are shown as dot plots before and after exposure to RHAMM or CD44 antibodies (FIG. 18A). Multiplexed detection of F-HA and the real-time interaction of F-HA with RHAMM and CD44 display are shown in FIG. 18B. Control cells (either untreated or incubated with non-immune IgG) grouped into the RHAMM−/CD44− quadrants as expected (FIG. 18Ai). Incubation of tumor cells with RHAMM antibodies resulted in staining above these background, control levels with the majority of cells exhibited a low intensity of staining. However, a few cells with high staining intensity were detected (FIG. 18Aii). MDA-MB-231 cells exhibited higher levels of CD44 than RHAMM, as indicated by flow profiles of anti-CD44 antibody binding (FIG. 18Aiii). These data are in agreement with our previous finding that MDA-MB-231 cells express both RHAMM and CD44 on their surface (Hamilton S. R., et al, JBC 2007, Biomacromolecule 2012]. The addition of F-HA to these cells resulted in the expected binding heterogeneity that we observed in FIG. 18 (FIG. 18Bi) which was not detected by either the CD44 or RHAMM antibodies (FIGS. 18Aii and 18Aiii).

Multiplexed analysis of RHAMM display and F-HA binding when cells exposed to F-HA prior to exposure to CD44 or RHAMM antibodies, show that display of both receptors was more heterogeneous after exposure to HA (F-HA). Most cells displaying CD44 bound to HA to a similar extent although a small number of cells those did not bind to F-HA (FIG. 18B, iii, +/− quadrant) and another small population displayed lower levels of CD44 (10³-10⁴ signal) although they bound to F-HA (FIG. 18B iii+/+quadrant,). These results show that all of the tumor cells express CD44 but not all of them bind to F-HA. However, the ability of cells to bind to F-HA appears to involve additional factors other than CD44 display since the outlier tumor cell populations that did not bound to F-HA expressed CD44 while other outliers that express relatively low levels of CD44 bind high levels of HA. Furthermore CD44 display was relatively homogeneous with or without addition of F-HA and so it alone does account for the binding heterogeneity of F-HA (FIG. 18B iii, HA⁺/CD44⁺). In contrast, most cells that bound to HA did not display RHAMM but all of the cells that displayed RHAMM also bound to F-HA (FIG. 18B ii, +/+ and −/+quadrants). Intriguingly, the addition of F-HA changed both the HA binding profiles and RHAMM display of tumor cells so that both F-HA binding and RHAMM display were more heterogeneous.

These data may suggest both that exposure of tumor cells to HA results in increased heterogeneity of cell surface RHAMM intensity and to a lesser extent CD44 and that these collectively can account for at least part of the heterogeneity of F-HA binding. However, the level of HA binding appears to be primarily related to CD44 display. This last observation is surprising since it indicates that while all of CD44 receptors are displayed on the surface, only a small number of RHAMM receptors are available on the surface for binding to HA. This is contrary to our previous observations that cell surface RHAMM is required for a motility response to HA in MDA-MB-231 tumor cells. We postulated that cell surface RHAMM might be rapidly internalized in response to HA exposure and might also mediate functions of HA inside the cell. We designed a series of simple experiments to test this possibility: (1) FACS analysis of F-HA binding cells confirmed internalization of F-HA at 37° C. as evidenced by significant reduction of second peak (FIG. 18C); (2) fluorescent microscopic analysis of the cells in culture, confirmed that cells took up F-HA and internalized it (FIG. 18D top). As expected from earlier data, the uptake was heterogeneous, however the cellular localization of F-HA was similar regardless of differences in uptake. Higher magnification images showed that HA accumulated at the cell membrane, particularly in cell processes that resembled invadopodia, as well as in the perinucleaus and nucleus (FIG. 18D bottom). (3) TEM analysis of cells with gold conjugated-HA appeared to indicate the presence of HA in the nucleolus (FIG. 18E), in agreement with uptake of fluorescent HA shown above; and (4) exposure of cells to a RHAMM mimetic peptide (Tolg et al) that interferes with the interaction of RHAMM with HA fragments (but not intact HA), prevented accumulation of F-HA in the nucleus without affecting perinuclear accumulation (FIG. 18F). Therefore RHAMM had a function in moving internalized F-HA into the nucleus.

We next assessed if F-HA binding heterogeneity is linked to phenotypic properties of tumor cells. We therefore sorted tumor cells according to their HA binding levels and monitored their proliferative, invasive and tumorigenic properties in 2D and 3D culture and in severely immune compromised mice.

Tumor Cell Subpopulations that Differ in their HA Binding Levels Exhibit Distinct Cellular Properties.

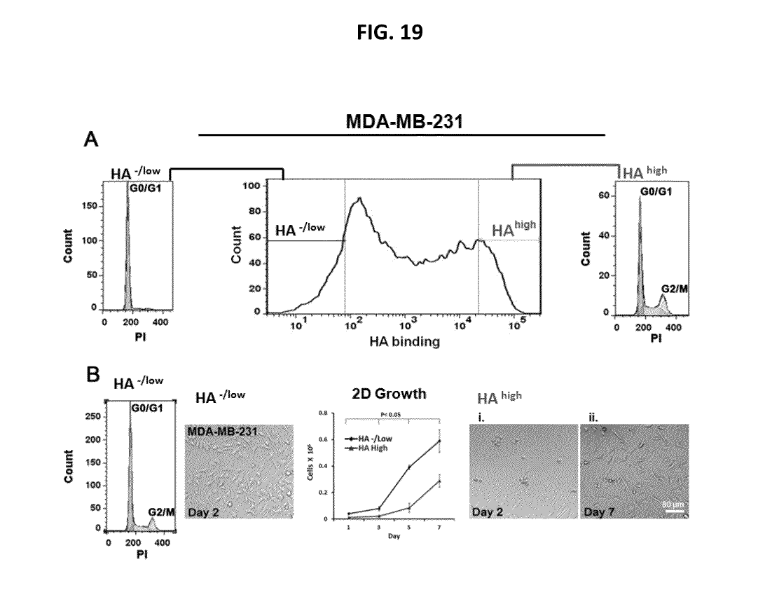

To gain insight into the nature and functions of the heterogonous subpopulations, we sorted and isolated HA^{-/low} and HA^{high} subpopulations based on differential binding of F-HA (markers indicate ˜8% selection) (FIG. 19A). We assessed cell cycle status, morphology and growth of isolated subpopulations under 2D and 3D culture conditions, as well as migratory capacity. Cell cycle assessment of freshly sorted HA^{high} subpopulation showed cell cycle distribution of G0/G1 (93.71%) and S phase (6.29%) for HA^{-/low} but G0/G1 (49.96%), S (32.98%) and G2/M (17.06%) for HA^{high}.

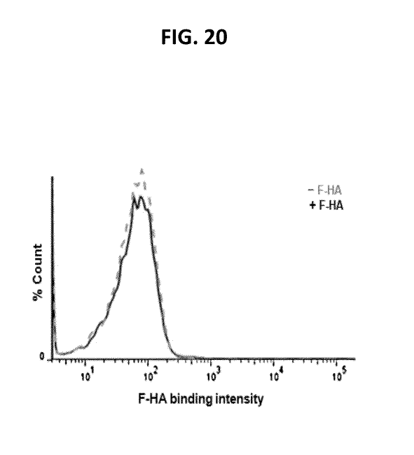

HA^{-/low} subpopulations grew more rapidly than HA^{high} subpopulations (FIG. 19). FIG. 19A is a schematic for identification and isolation of HA^{-./low} and HA^{high} subpopulations based on differential binding of cells to HA (markers indicate 8% selection) and their cell cycle modes immediately after isolation. Left side of FIG. 19B shows the morphology of HA^{-/low} subpopulations after adherence to 2D glass culture chambers within 2 days after sorting, right side of FIG. 19B shows the morphology of HA^{high} subpopulations on 2D culture chambers within 2 days (round cells were non-adherent initially) and 7 days after sorting, and the middle of FIG. 19B shows the growth status of subpopulations over 7 days. FIG. 20 depicts the fluorescent HA signal disappears by 7 days.

However, when these sorted cells were cultured in 2 dimensions, the HA^{-/low} subpopulation uniformly adhered to the substratum and proliferated rapidly so that by 2 days cells were almost confluent (FIG. 19B, left). Cell cycle analysis confirmed that these adherent HA^{low} tumor cells were proliferating in 2D culture (FIG. 19B, left). In contrast, the HA^{high} subpopulation, which remained round and clustered even after 2 days in culture, did not reach confluency until Day 7 (FIG. 19B, right panel). Thus, both HA^{high} and HA^{-/low} subpopulations proliferated in 2D and growth curve analyses confirmed that HA^{-/low} subpopulations grew more rapidly than HA^{high} subpopulations (FIG. 19B).

We next determined if F-HA would be retained on cell surface for long period of time, but FACS analysis showed that F-HA disappeared by 7 days (FIG. 20). We therefore performed colony formation assays in 2D and in 3D cultures expecting that HA^{low} would form larger colonies than HA^{high} in 2D but not 3D culture conditions. We performed the 3D colony assays using matrigel and soft agar (FIG. 21B,C) using limiting dilution to achieve colony growth from single cells. As predicted, HA^{-/low} formed larger colonies from single cells in 2D culture than HA^{high} tumor cells. However, HA^{-/low} tumor cells formed larger colonies in 3D matrigel and soft agar than HA^{high} tumor cells, showing that they are more able to proliferative in anchorage independent conditions than HA^{high} subpopulations. (FIG. 21B,C). These results suggest that binding of HA to tumor cells may induce a transient entry into cell cycle but this is not subsequently sustained. Rather, tumor cells that do not bind well to HA are unexpectedly more proliferative over time than those that bound high levels of this polysaccharide.

The morphology of HA^{high} colonies in 3D cultures were more heterogeneous and dispersed than HA^{-/low}, suggesting that they might be more invasive (e.g. FIG. 21B-C, right). To assess this, the invasiveness of both subpopulations were assayed in Matrigel coated-Boyden chambers (FIG. 21D). Results showed that HA^{high} subpopulations were approximately twice more invasive than their HA^{-/low} counterparts. We next utilized chick chorioallantoic membranes (CAM) to confirm these results in an in vivo setting.

Differential HA Binding Distinguishes Functionally Distinct Cell Subpopulations in MDA-MB-231 (FIG. 21).

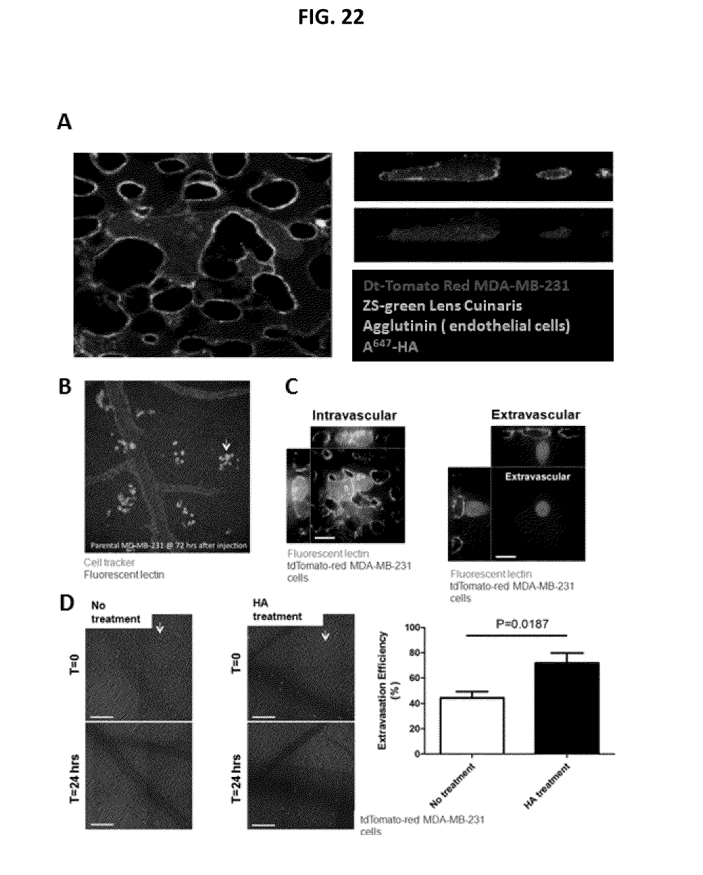

This model allows for real time observation of tumor cells as they flatten onto CAM vessel endothelia and extravasate. This allowed us to assess if cells that bind avidly to HA extravasate more efficiently than those that do not. MDA-MB-231 tumor cells were first mixed with F-HA and immediately injected into a CAM vein (FIG. 22A). The endothelial cells were marked with a green fluorescent lectin prior to the injection of tumor cells to visualize blood vessels. Real time confocal images showed that by 10-15 minutes after injection, F-HA was present within the blood vessels but had not been taken up into the interstitial spaces (blue, FIG. 22A). Tumor cells (red) initially rolled then flattened along the endothelium to arrest within the blood vessel. These cells had bound and internalized F-HA while cells that did not bind to F-HA did not roll or flatten onto the endothelium within the experimental time frame of xx hrs (FIG. 22A). Z-stacks of confocal images taken at 2 hrs after injection confirm the presence of F-HA (blue) vesicles inside tumor cells, which are surrounded by endothelium and thus have not yet extravasated into interstitial spaces. These results show that F-HA binding/uptake occurs in vivo and that it distinguishes tumor cells able to attach to vascular endothelium from those that cannot.

Extravasated tumor cells can be observed in interstitial spaces 24-72 hrs after their injection into CAM veins but by this time cell bound F-HA is not detectable since it has been metabolized (FIG. 22B). To determine if F-HA promotes extravasation of dt-Tomato expressing tumor cells, we therefore simply measured the number of extravasated tumor cells at varying times after injection with or without unlabeled HA, used at the same concentration and polydispersity as F-HA, with intravial confocal imaging (FIG. 22C-D). This approach also permitted us to assess if tagging HA influenced its bioactivity. We first confirmed that untreated MDA-MB-231 cells labeled with Cell Tracker can migrate through CAM endothelium and form viable interstitial tissue colonies. By 72 hrs, fluorescent tumor cell (green) colonies are observed surrounding blood vessels (red) as well as at a distance from blood vessels (FIG. 22C). These results confirm that this BCa tumor cell line is able to extravasate and form colonies within CAM tissue. We next determined if exposing tumor cells to HA increased extravasation, choosing 24 hrs after injection rather than 72 hrs shown in FIG. 22C to reduce the contribution of proliferation to appearance of extravascular tumor colonies. Confocal microscopic images were taken of x random fields and z stacks of these images were prepared to clearly differentiation between intra vs. extravascular tumor cells (FIG. 22D). The number of extravascular fluorescent tumor cells (red) with or without exposure to HA were counted from these images. Tumor cells exposed to HA extravasated with twice the efficiency of untreated cells. Collectively, these results show that tumor cells that bind to F-HA flatten and adhere to vessel endothelial cells as well as extravasate with enhanced efficiency compared to untreated (−F-HA) or tumor cells that bind low levels of HA.

HA^{High} Subpopulations Result in Slow-Growing but Locally Invasive Tumor Xenografts.

Our in-culture results suggested that HA^{high} supopulations are slowly proliferatve but highly invasive. We next determined if these properties were retained in vivo. Sorted MDA-MB-231 subpopulations, and unsorted parental tumor cells used as a control, were separately embedded in Matrigel and injected into the 4^{th} mammary fat pads of female NOD/LtSz-scid IL2Rgamma−/− mice. In agreement with in-culture data, HA^{-/low} subpopulations produced larger tumors than HA^{high} (FIG. 23). The tumor xenografts derived from HA^{-/low} cells (black) increased in size over an 8 week period at a rate similar to the unsorted, parental tumors (gray-dashed) but significantly faster than HA^{high} subpopulations (red). Wet weight measurements taken at 8 weeks confirmed that tumors derived from HA^{-/low} were similar in size to control, untreated parental tumor cells, and significantly larger than HA^{high} subpopulations. These in vivo data are consistent with in culture data and indicate that sorting CD44⁺/CD24⁻cells by their level of HA binding permits isolation of rapidly proliferating from slowly proliferating subpopulations. Since our in-culture data suggested that, despite their lower proliferation rate, HA^{high} subpopulations are more invasive than HA^{-/low}, we conducted a postmortem examination for local and distant dissemination of tumors. As shown in FIG. 23B, 16% of HA^{high} tumors invaded the mammary fat pad and peritoneum (marked by “T”). Neither unsorted parental tumor cells or HA^{-/low} sorted tumor cells exhibited gross evidence of local invasion (FIG. 23A,B). However, 9% of untreated control cells showed microscopic evidence of local invasion. No evidence of distant metastases in the lung or liver were observed in these same mice, although unsorted MDA-MB-231 BCa cells generally require a greater length of time and removal of the primary tumor for detectable metastasis formation to occur in these tissues. Pathological evaluation of tumor histology indicated that the unsorted parental and two sorted tumor subpopulations formed high grade adenocarcinomas. However, HA^{high} tumors contained spindle cell foci and evidence of perivascular invasion, lymphovascular invasion, and muscle invasion (FIG. 23B). These latter results confirmed the macroscopic evidence of lymphatic and muscle invasion. In contrast, HA^{-/low} tumors exhibited mainly discohesive epitheliod and necrotic phenotypes and, although 10% of them showed spindle cell foci and perivascular invasion, there was no evidence for lymphatic or muscle invasion (FIG. 23B). The unsorted parental tumors (control) exhibited an intermediate phenotype: they resembled HA^{-/low} tumors in that they contained large areas of discohesive epithelioid cells and necroses but, like HA^{high} tumors, also contained spindle cell foci and perivascular, muscle and lymphovascular invasion (FIG. 23B). Neverthetheless, the level of detectable invasion appeared to be greater in HA^{high} tumors than parental control tumors. To quantify this effect, we defined a local invasion index (LII) comprising the sum of perivascular invasion, lymphovascular invasion, and muscle invasion occurrence per cohort. HA^{high} tumors had a significantly higher LII (99%,) than either HA^{-/low} tumors (12%) or parental control tumors (63%). These results suggest that absent or low HA binding is associated with enhanced proliferation while high HA binding is linked to invasion.

Because CD44 cell-surface display correlated with HA binding level in both subpopulations, we next evaluated the CD44 expression of tumors by immunostaining Results indicated highly heterogeneous patterns within and between tumors, with the HA^{high} and parental line (control) being more morphologically heterogeneous than the HA^{-/low} tumors. The staining was prominent at the tumor stromal edge for both control and HA^{high} derived tumors (FIG. 23C, top panel), and they seemed to contain a capsule of modified myofibroblasts as opposed to HA^{-/low}. Notably, cell-surface CD44 was higher in HA^{high} than either the HA^{-/lows} or controls, and they appeared to have more ECM, as the cells did not nest together as much as the HA^{-/low} or controls cells did (FIG. 23C, bottom panel). HA staining in tumors was highly heterogeneous and quantification of staining showed a trend for increased HA accumulation in tumors derived from HA^{high} subpopulations, but this did not reach statistical significance.

These results established that sorting parental MDA-MB-231 cells on the basis of HA binding enriched for a minor heterogeneous subset that was poorly proliferative but highly invasive.

In its native form, HA is a large but polydisperse glycosaminoglycan, whose molecular weight in tissues varies from several hundred kDa to over 20 mD. It performs a variety of homeostatic functions including hydration, sequestering growth factors, providing a visco-elastic environment for tissues and regulating innate immunity [Järveläinen H, 2009 Extracellular matrix molecules: potential targets in pharmacotherapy Pharmacol Rev 61: 198-223]. During tissue repair and disease, in particular diseases with an inflammatory component, HA is reduced to fragments (e.g. 40-300 kDa) and oligosaccharides (<40 kDa) by hyaluronidases released by secretion or by dying cells. These fragments activate innate immune cells and promote inflammation and also promote the migration, invasion, extravasation and plasticity of tumor cells including BCa. HA binds to a number of receptors including CD44, RHAMM and LYVE1, all of which have been implicated in BCa. To date, CD44 and RHAMM are the most extensively studied HA receptors in BCa aggression. CD44 is subject to extensive alternative splicing, which can theoretically give rise to 800 variants (Naor). Variant expression modifies the binding of HA to CD44 by allosteric mechanisms, so depending on the variant that is expressed, CD44 can bind to and utilize HA as a substrate to accomplish a wide variety of cell functions. Modulation of CD44/HA binding affinity permits weak adhesion to endothelial cells necessary for the “rolling” of tumor cells that is required for their successful extravasation. Alternating weak/strong adhesions between CD44 and HA permit motility. In addition, CD44/HA interactions stimulate the formation of invadopodia, which are sites where proteolytic enzymes such as MMP's are released and which are required for invasion of tumor cells through tissues.

The two HA receptors expressed by malignant breast tumors are CD44 and RHAMM. CD44 is a type I integral transmembrane glycoprotein receptor that binds to HA via an extracellular domain and partners with the unconventionally exported mitotic spindle and hyaluronan binding protein, RHAMM/HMMR to regulate activation of signaling pathways that promote tumor cell invasion and metastasis. Role of CD44 in BCa progression is controversial and although CD44 targeting can markedly reduce malignant activity in a number of animal models, the validity of CD44 as a diagnostic or therapeutic target in human malignant diseases is not certain. Recently, some studies suggest that CD44⁺/CD24^{-/low} subpopulations enhance invasive properties and favor distant metastasis; as such, CD44-expressing breast cancer cells with ‘stem-like’ characteristics are present in the bone marrow of BCa patients with early-stage disease. It has been proposed that the conflicting roles of CD44 in experimental models of tumorigenesis may be due to the presence or absence of its RHAMM/HMMR partner [Maxwell, C. A., J. McCarthy, et al. (2008). “Cell-surface and mitotic-spindle RHAMM: moonlighting or dual oncogenic functions?” J Cell Sci 121(Pt 7): 925-932], which is commonly overexpressed in many advanced cancers and its hyper-expression in primary breast tumor subsets predicts poor clinical outcome and increased peripheral metastasis (Wang, Thor et al. 1998; Pujana, Han et al. 2007). We have previously shown that disruption of CD44, HA, and RHAMM interactions blocked invasion of breast cancer cells (Hamilton, Fard et al. 2007) and propelled them into apoptosis suggesting that this complex may provide important survival and migration cues that contribute to breast cancer progression [Kimata et al. Cancer Res—1983; 43:1347-1354].

Our developed method for profiling HA binding and real-time multiplexed analysis of HA/CD44/RHAMM interaction, revealed a novel form of tumor heterogeneity to predict aggressive tumor behavior. Multiplexed analysis indicate that the majority of the RHAMM that was not on the surface may have a function in moving the HA into the nucleus. It suggested further that the observed heterogeneity, was not due to the heterogeneity of cellular receptors but was due to the inherent heterogeneity of the cells to bind to HA. To understand the complete landscape of tumor heterogeneity may be difficult, however what is important is to identify the population(s) that confer aggressive behavior on the tumor because identifying and targeting these populations which are key in inducing metastasis will directly impact disease classification, prognosis, and therapy.

We showed that HA binding to BCa cancer cells revealed a significant heterogeneity with respect to cell morphology, proliferation rate, and invasive ability. In a surrogate of metastatic and triple-negative breast cancer (ER−/PR−/HER2−) BCa subtype, high level of HA binding (HA^{high}) was associated with invasive phenotype of a minor subpopulation. To quantify and obtain a comparable index of heterogeneity we defined heterogeneity index (Het.I) and showed that highly metastatic cell lines had a higher Het.I. This allows clinicians to sort patients with primary cancers and identify those patients at risk for metastases and those patients whose cancer is not or less likely to metastasize. Moreover, using Het.I for assessments of tumor heterogeneity as part of a diagnostic may facilitate better prediction of a patient's potential for metastasis, and provide rationale and selection for therapy regimens that increase the response rates of patients with relapse.

To obtain information on spatial distribution of heterogeneity as a function of invasive phenotypes, we defined the local invasion index (LII) as sum of perivascular invasion, lymphovascular invasion, and muscle invasion in vivo. This simple index provided a focal measure of a subpopulation's heterogeneities and allowed us to segregate the drivers of tumor metastasis from growers. Although some of these results are in agreement with studies that reported substantial correlation between high cell-surface HA and HAS2 gene expression with migratory capacities and distant metastasis, the slow proliferation of invasive subpopulations challenges the current paradigm of fast growing tumors lead metastasis. These findings suggest that HA and its derivatives could be exploited for diagnosis and therapy of invasive BCa subpopulations.

Furthermore, the tumor cells sorted into either HA^{high} or HA^{low} subpopulations but were not considered to comprise stem or progenitor cells because they did not meet the gold standard used to define “tumor cell stemness”, i.e., the tumor stem cell or tumor initiating cells should give rise to the same heterogeneity as the tumor it was derived from. Thus, if either HA^{high} or HA^{low} subpopulations were stem or progenitor cells they would grow like the parental tumor from which they arose. However, as seen in FIG. 23, HA^{high} subpopulations are much more invasive and less proliferative than the parental tumors while HA^{low} tumors are much less invasive.

### Example 5

### Prognosis and Influencing Therapy by Detecting Cells Actively Metabolizing Hyaluronan

Unexpectedly, we found that HA^{high} subpopulations were proliferating slowly While classical chemotherapy has successfully targeted the highly proliferating population of many tumor cells, the slowly proliferating tumor cells which are nowadays accepted as the major cause of relapse are often left behind. Our HA binding assay may provide a means to devise detection and treatment strategies capable of adapting to altered population growth rates in addition to altered invasive phenotypes. Our finding that high HA binding predicts attenuated growth of a minor subpopulation of BCa tumor cells supports reports that high cellular HA coat formation and intratumoral HA accumulation within tumors contribute to ‘chemotherapy resistance’—a function that is shared by slow-growing cells tumor (or dormant cancer cells) that are nowadays accepted as major causes of cancer relapse.

These findings suggest that HA and its derivatives could be exploited for diagnosis and therapy of invasive BCa subpopulations. Development of diagnostics that are based upon the molecular characteristics of aggressive subtypes would offer the additional advantage of sorting patients into different treatment streams to reduce patient morbidity and medical costs, and to increase effective clinical outcome. The target-binding capacity of these HA-based probes may be exploited to target chemotherapy or toxic agents directly to the site of the invasive tumor tissue. That is, these HA-based agents can function as “theranostics,” in which cancer treatments are tailored for delivery to the tissue type of individual patients, while the results of that intervention are simultaneously assessed by the diagnostic component.

The above examples are provided to illustrate the invention but not to limit its scope. Other variants of the invention will be readily apparent to one of ordinary skill in the art and are encompassed by the appended claims. All publications, databases, and patents cited herein are hereby incorporated by reference for all purposes.

TABLE 1

| | | | | Rhamm | |
|---|---|---|---|---|---|
| | Molecular | Tumor | Cancer stem-like | Surface | HA |
| Cell Lines | subtypes | phenotype | surface marker | marker | binding |
| MDA-MB- | Basal | Highly invasive | CD44⁺/CD24^{-/low} | Rham⁺ | Very high |
| 231 | | | | | |
| T4-2 | Basal | Weakly invasive | CD44⁺/CD24^{low} | Rhamm⁺ | High |
| SI | Basal | Non-malignant | CD44⁺/CD24^{low} | Rhamm⁻/^{low} | Medium |
| MCF-10A | Basal | Non-malignant | CD44⁺/CD24⁺ | Rhamm⁺ | Medium |
| MCF-7 | Luminal | Weakly invasive | CD44^{low}/CD24⁺ | Rhamm⁺⁺ | Low |
| SKBR-3 | Luminal | Weakly invasive | CD44⁻/CD24⁺ | Rhamm⁺⁺ | Low |

TABLE 2

| Histological phenotype | HA^{Neg/Low binding} | HA^{High binding} |
|---|---|---|
| Tumor Grade | High | High |
| Muscle invasion | − | + 2/6^{¥} |
| Fat infiltrating | + | + |
| Focal spindle cell | − | ++ 3/6, + 2/6,−⅙ |
| Lymphovascular invasion | − | + 2/6 |
| Invasive adenocarcinoma | + | + |
| Discohesiveness | + | ++⅙,+ 2/6,− 3/6 |
| Focal de-differentiation | + 2/6 | − |
| Tumor necrosis | + 2/6 | − |

^{¥}gross assessment was confirmed by microscopy

TABLE 3

| | Control | | |
|---|---|---|---|
| | (Untreated/Unsorted) | HA^{Neg/Low} | HA^{High} |
| Histological Phenotype | (n = 11) | (n = 10) | (n = 12) |
| Lymphovascular invasion | ++ | − | +++ |
| | (36%) | | (58%) |
| Perivascular invasion | + | + | ++ |
| | (18%) | (10%) | (25%) |
| Muscle invasion | + | − | + |
| | (9%) | | (16%) |
| Spindle cell foci | ++ | + | ++++ |
| | (36%) | (10%) | (75%) |
| Discohesive epithelioid | ++++ | ++++ | ++ |
| | (100%) | (90%) | (33%) |
| Necrotic foci | +++ | +++ | − |
| | (72%) | (50%) | |

+: 0%-25% occurrence
++: 25%-50% occurrence
+++: 50%-75% occurrence
++++: 75%-100% occurrence

TABLE 4

| # | Cell Lines | Type | CD44 | CD24 | HA | ER | PR | HER2 | 3D Morphology |
|---|---|---|---|---|---|---|---|---|---|
| 1 | MCF-10A | Basal B | + | | | − | − | +/_WT | |
| 2 | S1 | Basal B | + | − | + | | | | Round |
| 3 | T4-2 | Basal B | + | | | | | | Mass |
| 4 | MDA-MB-231 | Basal B | ++ | − | +++ | − | − | | Stellate |
| 5 | SUM1315MO2 | Basal B | + | + | ++ | − | − | | |
| 6 | HS-578T | Basal B | +++ | − | ++ | − | − | | Stellate |
| 7 | MDA-MB-468 | Basal A | ++ | + | | − | − | | Grape-like |
| 8 | HCC-1569 | Basal A | + | − | + | − | − | + | Mass |
| 9 | BT-20 | Basal A | ++ | | | − | − | | |
| 10 | MCF-7 | Luminal | + | + | ++ | + | + | | Mass |
| 11 | BT474 | Luminal | − | + | | + | + | + | Mass |
| 12 | SK-BR3 | Luminal | − | <+ | + | − | − | + | Grape-like |
| 13 | AU565 | Luminal | | | | − | − | + | Grape-like |
| 14 | MDA-MB-361 | Luminal | − | <+ | + | + | − | + | Grape-like |

TABLE 5

| # | Cell Lines | Type | CD44 | RHAMM | HA | ER | PR | HER2 |
|---|---|---|---|---|---|---|---|---|
| 1 | MCF-10A | Basal B | | | | − | − | +/_WT |
| 2 | S1 | Basal B | | | | | | |
| 3 | T4-2 | Basal B | ++ | + | ++ | | | |
| 4 | MDA-MB-231 | Basal B | +++ | + | ++++ | − | − | |
| 5 | SUM1315MO2 | Basal B | + | | +++ | − | − | |
| 6 | HS-578T | Basal B | ++++ | | +++ | − | − | |
| 7 | MDA-MB-468 | Basal A | | | | − | − | |
| 8 | | Basal A | | | | − | − | + |
| 9 | BT-20 | Basal A | ++ | | + | − | − | |
| 10 | MCF-7 | Luminal | + | ++ | + | + | + | |
| 11 | BT474 | Luminal | − | | + | + | + | + |
| 12 | SK-BR3 | Luminal | − | ++ | + | − | − | + |
| 13 | AU565 | Luminal | | | | − | − | + |
| 14 | MDA-MB-361 | Luminal | − | | + | + | − | + |

TABLE 6

| | HA binding level | HA receptor | display | Subtype |
|---|---|---|---|---|
| MDA-MB- | HA⁴⁺ | CD44³⁺ | RHAMM⁺ | Basal |
| 231 | | | | |
| (ER⁻,PR⁻, | | | | |
| HER2⁻) | | | | |
| T4-2 | HA²⁺ | CD44²⁺ | RHAMM⁺ | Basal |
| (ER⁻,PR⁻, | | | | |
| HER2⁻) | | | | |
| MCF-7 | HA | CD44^{low/+} | RHAMM²⁺ | Luminal |
| (ER+,PR⁺,H | | | | |
| ER2⁻) | | | | |
| SKBR-3 | HA⁺ | CD44⁻ | RHAMM²⁺ | Luminal |
| (ER⁻,PR⁻, | | | | |
| HER2⁺) | | | | |

What is claimed is:

1. A method for isolating a cell, comprising: (a) providing a polydisperse (5-500 kDa Molecular weight range) labeled hyaluronan probe (HA probe); (b) contacting a cell with the labeled HA probe; (c) detecting high HA uptake in said cell, wherein high HA uptake is at least 100 fold differences in HA binding and/or uptake and metabolism by said cell, which indicates said cell is highly invasive, slow-growing and able to invade the lymphovasculature, and (d) isolating said cell having high HA uptake.

2. The method of claim 1, wherein said cell having a genotype of CD44⁺/CD24⁻/RHAMM⁺/HA^{high}.

3. A method of prognosis of a cancer patient comprising the steps of: (a) obtaining a tissue biopsy from a patient; (b) labeling cells from said biopsy with a labeled hyaluronan probe; (c) sorting said cells based on high or negative/low uptake of the labeled hyaluronan probe; (d) determining if any cells highly uptake the labeled hyaluronan probe, wherein high uptake of the labeled hyaluronan probe by cells indicates those cells are highly invasive and able to invade the lymphovasculature.

4. The method of claim 3, wherein the biopsy is obtained from an epithelial tissue.

5. The method of claim 4, wherein the epithelial tissue is breast tissue.

6. A method of inhibiting cancer cell metastasis, the method comprising: (a) providing a polydisperse (5-500 kDa Molecular weight range) labeled hyaluronan probe (HA probe) attached to a therapeutic; (b) administering the labeled HA probe-therapeutic to a subject; (c) detecting high HA uptake in a cell, wherein high HA uptake is at least 100 fold differences in HA binding and/or uptake and metabolism by said cell, which indicates said cell is invasive, slow-growing and able to invade the lymphovasculature.

7. The method of claim 6 wherein the therapeutic is an antibody, a small molecule, a mimetic, a peptide, a siRNA, an antisense oligo, or an aptamer.

8. A probe for targeting triple negative breast cancer cells and delivering therapy to those cells, said probe comprising a polydisperse (5-500 kDa Molecular weight range) hyaluronan probe (HA probe) attached to a therapeutic.

9. A probe for identifying tumorigenic cell populations, comprising a HA-binding targeting component and an imaging component, wherein said HA-binding targeting component specifically binds to the CD44⁺/CD24⁻/RHAMM⁺/HA^{high} cells, and wherein said imaging component is a detectable label.

10. The probe of claim 9 wherein the ligand is hyaluronan, a hyaluronan fragment, or any chemical composition that is an HA mimetic.

11. The probe of claim 9 wherein the detectable label is a metal or fluorescent label.
